(19) United States
(12) Patent Application Publication
(10) Pub. No.: US 2025/0139336 A1
(43) Pub. Date: May 1, 2025

(54) METHODS FOR ANALYSES OF INTERNAL STRUCTURES AND DEFECTS IN MATERIALS USING PHYSICS-INFORMED NEURAL NETWORKS

(71) Applicants: NANYANG TECHNOLOGICAL UNIVERSITY, Singapore (SG); BROWN UNIVERSITY, Providence, RI (US); Massachusetts Institute of Technology, Cambridge, MA (US)

(72) Inventors: Subra SURESH, Singapore (SG); George Em KARNIADAKIS, Newton, MA (US); Enrui ZHANG, Providence, RI (US); Ming DAO, West Roxbury, MA (US)

(73) Assignees: NANYANG TECHNOLOGICAL UNIVERSITY, Singapore (SG); BROWN UNIVERSITY, Providence, RI (US); Massachusetts Institute of Technology, Cambridge, MA (US)

(21) Appl. No.: 18/836,071

(22) PCT Filed: Feb. 6, 2023
(86) PCT No.: PCT/US2023/012420

§ 371 Date: Aug. 6, 2024

Related U.S. Application Data
(60) Provisional application No. 63/307,478, filed on Feb. 7, 2022.

(51) Int. Cl. G06F 30/27; G06N 3/08; G16C 60/00

(52) U.S. Cl. CPC G06F 30/27; G06N 3/08; G16C 60/00

(57) ABSTRACT

Methods involving physics-informed deep learning to help solve inverse problems of solid materials/structures related to unknown geometry include (1) identifying and characterizing unknown materials/structures and defects with accuracy and predictive capability and limited non-destructive measurements, and/or (2) designing geometrical features and parameters of solid materials and structures to achieve optimized and/or improved performance.

### CROSS-REFERENCE TO RELATED APPLICATIONS

The present application claims the benefit of U.S. provisional application No. 63/307,478, filed Feb. 7, 2022, the entire contents of which is incorporated herein by reference.

### FIELD OF THE INVENTION

The present invention is related to physics-informed machine learning, and, in particular, utilizing physics-informed neural networks to examine internal structures and defects of solid materials, including (1) geometry identification and (2) design of optimized and/or improved geometry.

### BACKGROUND

Deep learning approaches play an increasingly significant role in a wide range of technologies that benefit computer vision, natural language processing, and other data-rich areas of societal interest. Despite the evolving sophistication of data analytics and neural networks (NNs), much of this work to date has not been predicated on a large volume of scientific data, through which predictive models can be constructed using experimentally validated mechanistic inferences and laws of physics. In most scientific applications, by contrast, physical conservation laws (such as those for momentum and energy) are framed by highly general, mathematical formulations (e.g., those invoking partial differential equations (PDEs) in areas such as solid mechanics, fluid mechanics, and material diffusion), along with experimental authentication by recourse to laboratory tests.

### SUMMARY

According to one aspect, a method for analyzing an aspect of a solid material/structure is provided. The method includes receiving one or more geometric variables as one or more inputs to physics-informed neural networks (PINNs). The method also includes characterizing/parametrizing a first geometry according to the one or more geometric variables. The method further includes identifying one or more aspects of the solid material/structure based on the first geometry, wherein characterizing/parametrizing a first geometry is performed in a trainable manner.

According to another aspect, a method for analyzing an aspect of a solid material is provided. The method includes receiving one or more geometric variables as one or more inputs to physics-informed neural networks (PINNs). The method also includes generating features and parameters of a second geometry of one or more aspects of a solid material, wherein generating features and parameters of a second geometry achieves an improved performance based on one or more objectives, wherein an improved performance based on one or more objectives is performed in a trainable manner.

In some exemplary embodiments of the methods, the one or more aspects of the solid material/structure includes at least one of one or more internal structures, internal surfaces/boundaries, external structures, or external surfaces/boundaries. In some exemplary embodiments of the methods, the one or more aspects of the solid material/structure includes one or more defects in the solid material/structure.

In some exemplary embodiments of the methods, the trainable manner includes substituting one or more geometric variables such as geometry trainable variables, geometry-dependent training points, and/or making the gradient with respect to geometry tractable.

In some exemplary embodiments, characterizing/parametrizing a first geometry includes inversely characterizing a first geometry of at least one of one or more internal structures, internal surfaces/boundaries, external structures, or external surfaces/boundaries according to displacement data. In other exemplary embodiments, generating features and parameters of a second geometry includes inversely designing the second geometry of at least one of one or more internal structures, internal surfaces/boundaries, external structures, or external surfaces/boundaries according to a predefined objective function.

In some exemplary embodiments, identifying one or more aspects of the solid material/structure based on the first geometry includes concurrently identifying one or more full field stresses, strains, and/or displacements in the one or more aspects of the solid material/structure.

In some exemplary embodiments of the methods, a framework for inversely characterizing and/or designing involves unknown/moving domains directly parameterizing a computational domain/geometry with material and geometry parameterization. In some exemplary embodiments, inversely characterizing a first geometry includes minimizing a discrepancy/loss between the displacement data and one or more results of a forward solver. In other exemplary embodiments, inversely designing a second geometry includes minimizing the objective function.

In some exemplary embodiments of the methods, characterizing/parametrizing a first geometry or generating features and parameters of a second geometry includes representing of at least one of one or more internal structures, internal surfaces/boundaries, external structures, or external surfaces/boundaries by analytical function(s), parameterized function(s), a non-uniform rational basis spline (NURBS) or other neural network(s). Additionally, a shape of the at least one of one or more internal structures, internal surfaces/boundaries, external structures, or external surfaces/boundaries are simple or arbitrarily complicated.

In some exemplary, embodiments, concurrently identifying the full field stresses, strains, displacements in the one or more solid materials/structures includes one or more different shapes and/or topologies of the one or more solid materials/structures and different constitutive models for describing the mechanical properties of the one or more solid materials/structures. Further, the different constitutive models for describing the mechanical properties of the one or more solid materials/structures include measuring linear elasticity, nonlinear elasticity or hyperelasticity, and plasticity.

In some exemplary embodiments of the methods, the trainable manner includes a pretraining process for the PINNs. This pretraining process includes maintaining one or more estimated unknown parameters θ defined as fixed/not trainable and updating one or more trainable parameters of a neural network (NN) λ for one or more iterations. The pretraining process also includes solving one or more forward problems to capture a qualitative pattern of a displacement field and a stress field. The pretraining process further includes solving one or more forward problems until both a loss function and one or more estimated geometric parameters reach a relative plateau following pretraining of the PINNs. Additionally, both λ and θ are then trainable, with λ converged towards to a desired local minimum, and the pretraining process stabilizes the trainable manner of the PINNs. In some exemplary embodiments, an estimation of geometric parameters is automatically updated as the PINNs minimize the loss function during the pretraining and/or training process for the PINNs, including enforcing one or more diverse types of conditions in problem definition for integration into the PINNs in the form of the loss function during the pretraining and/or training process for the PINNs.

In some exemplary embodiments of the methods, the one or more geometric variables parameterize the computational domains of partial differential equations (PDEs) and boundary conditions. Additionally, the one or more geometric variables are first defined as trainable before expressing one or more locations of residual points as functions of the one or more geometric variables. In some exemplary embodiments, the one or more locations of the residual points are automatically updated as an estimation of the one or more geometric variables are updated throughout the training process. Additionally, the one or more locations of the residual points for one or more different conditions are in their correct domains, allowing the capturing of a gradient of a loss function [symbol] with respect to the one or more geometric variables. Further, the residual points for the one or more different conditions allows for the PINNs to correctly update estimation and/or design of the one or more geometric variables throughout the training process and characterize/design the at least one of one or more internal structures, internal surfaces/boundaries, external structures, or external surfaces/boundaries and/or defects.

In some exemplary embodiments, characterizing/parametrizing a first geometry includes accurately estimating the one or more geometric variables and one or more material parameters with limited non-destructive measurements. Accurately estimating the unknown geometric and one or more material parameters includes a relative error O(10⁻²) when proper displacement data are supplied to ensure identifiability. Additionally, characterizing/parametrizing a first geometry further includes placing one or more displacement measurement points only on an boundary of a solid material/structure.

According to another aspect, a method for utilizing physics-informed neural networks (PINNs) to examine internal structures and defects of solid materials/structures is provided. The method includes applying a neural network to approximate the primary solution fields. The method also includes integrating one or more mechanical laws into the PINN by deriving relevant mechanical quantities of interest from one or more neural network (NN) outputs, such as strain, stress, and/or residual of equilibrium partial differential equations (PDEs). The method further includes formulating a loss function [symbol] (λ, θ), wherein the loss function [symbol] (λ, θ) measures a discrepancy between predicted mechanical quantities of interest and their respective true values provided by the one or more mechanical laws and measured data. The method further includes conducting parameter estimation through a training of the PINN, wherein the training of the PINN includes updating/training unknown parameters θ=(θₘₐₜ, θ_{geo}) and neural networks parameters λ to minimize the loss function.

According to another aspect, a method for utilizing physics-informed neural networks (PINNs) to examine internal or external structures and defects of solid materials/structures is provided. The method includes minimizing [symbol] _{PDE}(λ, θ), [symbol] _{BC}(λ, θ), wherein minimizing [symbol] _{PDE}(λ, θ), [symbol] _{BC}(λ, θ) includes satisfying a governing partial differential equation (PDE) and one or more boundary conditions as the PINN seeks to minimize a loss function. Additionally, one or more constraints are satisfied through minimizing [symbol] _{cnstr}(λ, θ), wherein the one or more constraints are directly incorporated through designing an architecture of PINNs. Further, a design target is achieved through minimizing [symbol] _{target}(λ, θ), and one or more relevant geometric parameters in θ are adjusted to minimize the loss function and realize a design of optimal geometry.

### BRIEF DESCRIPTION OF THE DRAWINGS

The present disclosure will be described more fully hereinafter with reference to the accompanying drawings, in which preferred embodiments of the disclosure are shown. This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like numbers refer to like elements throughout. Other objects, features and advantages of the present disclosure will become apparent from the detailed description of the disclosure, which follows when considered in light of the accompanying drawings in which:

FIG. 1A provides a diagram of an architecture of the PINN for a plane-strain problem for a compressible linear elasticity material model.

FIG. 1B provides a diagram of an architecture of the PINN for a plane-strain problem for an incompressible hyperelasticity material model.

FIG. 1C provides a diagram of an architecture of the PINN for a plane-strain problem for a compressible deformation plasticity material model.

FIG. 1D a tabular view of definitions of mechanical quantities of interest in the architectures of PINNs for inverse problems in continuum solid mechanics including geometry identification and geometry design according to the present disclosure.

FIG. 2 provides charts of the placement of residual points for the initial geometry and updated geometry in Cases 0, 4 and 7B.

FIG. 3 provides a diagram of the general setup of Cases 0-5 of the prototypical problem on geometry and material identification in the present disclosure.

FIG. 4A provides a diagram of the specific setup of Case 0 evaluating compressible linear elasticity for one elliptical void as relates to geometry and material identification in the present disclosure.

FIG. 4B provides four diagrams of the specific setup of Cases 1, 2, 4, and 5, evaluating incompressible Neo-Hookean hyperelasticity for one elliptical void, one slit-shaped void, two circular voids, and one circular inclusion, respectively, as relates to geometry and material identification in the present disclosure, and also includes the legend relating to respective sketching schemes includes in FIGS. 3-8.

FIG. 4C provides a diagram of the specific setup of Case 3 evaluating compressible deformation plasticity for one circular void as relates to geometry and material identification in the present disclosure.

FIG. 4D provides a diagram of the respective hashing schemes for FIGS. 3-8 relating to void, matrix, and inclusion problems of linear elasticity, hyperelasticity, and deformation plasticity.

FIG. 5 provides a tabular view of parameter estimation for Cases 0-5 shown in FIGS. 3 and 4.

FIG. 6 provides six diagrams of the inference of the deformed patterns compared with ground truth (obtained from FEM) for Cases 0-5, shown in FIGS. 3 and 4A-4C.

FIG. 7 provides an illustration of the inference of the stress patterns compared with ground truth (obtained from FEM) for Case 4 shown in FIGS. 3 and 4.

FIG. 8 provides an illustration of the inference of the plastic zone compared with ground truth (obtained from FEM) for Case 3 shown in FIGS. 3 and 4.

FIG. 9 provides a graphical depiction of the setup of Case 6 on a fiber-reinforced material.

FIG. 10 provides a graphical depiction of the designed fiber thickness profile in Case 6 shown in FIG. 9.

FIG. 11 provides a tabular view of the design results compared with the optimal reference solution (obtained from FEM) in Case 6 shown in FIG. 9.

FIG. 12 provides graphical depictions of the setup of Cases 7A and 7B on a matrix-void system.

FIG. 13 provides a graphical depiction of the designed void shape in Case 7A shown in FIG. 12.

FIG. 14 provides a graphical depiction of the optimal reference solution (obtained from FEM) in Case 7A shown in FIG. 12.

FIG. 15 provides a tabular view of the design results compared with the optimal reference solution (obtained from FEM) in Case 7A shown in FIG. 12.

FIG. 16 provides a graphical depiction of the designed void shape in Case 7B shown in FIG. 12.

FIG. 17 provides a graphical depiction of the setup of Case 7C on a matrix-void system with multiple loading conditions.

FIG. 18 provides a tabular view of the design results compared the optimal reference solution (obtained from FEM) in Case 7C shown in FIG. 17.

FIG. 19 provides a graphical depiction of the setup of Case 8 on a simple bridge.

FIG. 20 provides a graphical depiction of the designed results compared with the optimal reference solution (obtained from FEM) in Case 8 shown in FIG. 19.

FIG. 21 provides a tabular view of the design results compared with the optimal reference solution (obtained from FEM) in Case 8 shown in FIG. 19.

FIG. 22 provides the parameter estimation results in Case 4 shown in FIGS. 3 and 4 with different strategies of optimizers.

FIG. 23 provides a diagram illustrating the geometry of the curved beam relating the multiple loading conditions illustrative example (Case 9).

FIG. 24 provides a graph illustrating the evolution of the designed parameter introduced in FIG. 23.

FIG. 25 provides a graph illustrating the force-strain curve of the designed geometry predicted by the PINN and the ground-truth curve introduced in FIG. 23.

### DETAILED DESCRIPTION

Aspects of the present disclosure include methods involving physics-informed deep learning to help solve inverse problems of solid materials/structures related to unknown geometry. Practically, this method helps to (1) identify and characterize unknown materials/structures and defects at great levels of accuracy and predictive capability with limited non-destructive measurements, and (2) design geometrical features and parameters of solid materials and structures to achieve optimized and/or improved performance.

Emerging research reveals the profound untapped potential of physics-based, multidisciplinary, deep learning approaches with unprecedented opportunities for scientific and engineering advances in molecular analysis, design of materials with improved properties and performance in structural and functional applications, and unique pathways for the characterization of properties of materials. To further realize this potential, broadly applicable methodologies in the area of NNs are needed to address a variety of issues that underpin deep learning analyses, governed by physical laws and guided by mathematical formulations. To this end, a physics-informed deep learning approach has recently been proposed for the simulation of systems governed by physical laws that are represented by PDEs. While traditional methods based on deep learning encode such formulations implicitly by feeding training data governed by equations, this approach explicitly encodes known physical or scaling laws in the form of mathematical equations into the standard structure of NNs, formulating the so-called Physics-Informed Neural Networks (PINNs). Such an approach integrates any existing knowledge expressible in terms of PDEs during the learning process, thereby markedly improving predictability while reducing the amount of data required to achieve a desired level of accuracy. Studies have shown the applicability of PINNs in addressing a wide spectrum of forward and inverse problems spanning disciplines such as fluid mechanics, quantum mechanics, and solid mechanics. Such applications have shown promise for enhancing predictability when the amount of data is limited or when the problem is ill-posed, situations in which existing methods are not likely to yield accurate and reliable results. This approach has been further extended to offer new pathways to address relevant mathematical formulations, such as stochastic PDEs and fractional PDEs.

Aspects of the present disclosure address this urgent need and overcome the various disadvantages of previously known systems and methods by addressing geometry identification and geometry design problems in the broad field of continuum solid mechanics with potential applications in many branches of engineering, sciences and medicine. In a geometry identification problem, the unknown geometric features and parameters are determined in a solid material/structure given measured material response under static or dynamic loading, thereby characterizing unknown structures including internal defects or boundaries such as voids, vacancies or holes, inclusions and reinforcements, and/or cracks. In a geometry design problem, one needs to identify the optimal geometric feature so that the performance of the material/structure is optimized and/or improved under certain metrics. Traditionally, computational algorithms for geometry identification/design are established based on the finite element method (FEM) as the forward solver. Beyond the forward solver, considerable effort is required for the design and implementation of iterative algorithms for updating the estimated/designed values of geometric parameters. Through the iterative algorithm, for identification problems, the discrepancy (loss) between the observed data and the results of the forward solver is minimized; for design problems, the objective function is minimized. However, the embedded forward FEM solver as a mesh-based method inherently brings about complications in these algorithms. The estimated/designed geometry is updated by repeatedly remeshing the domain through iterations. Alternatively, the unknown domain is embedded in a larger fixed domain while introducing an auxiliary field to track the presence of material. The problem becomes even more challenging when large deformations (i.e., geometric nonlinearity) and nonlinear mechanical properties (i.e., highly nonlinear constitutive behavior of the solid material) are involved. Available methods are cumbersome and resource-intensive for deriving automated solutions to such inverse problems involving unknown geometry.

The present disclosure relates to a novel method based on physics-informed machine learning, and, in particular, utilizing physics-informed neural networks to examine internal structures and defects in materials. It is noted that these unknown internal structures and defects can also be referred to as aspects. It is also noted that these materials can be solid or non-solid, despite illustrative embodiments treating the materials as primarily solid. This method solves inverse problems related to unknown geometry, which in practice can be applied to (1) identify and characterize unknown internal structures and defects with limited non-destructive measurements, and (2) design geometry of solids to achieve optimized and/or improved mechanical performance including maximizing or minimizing individually or in certain combinations the stresses, strains, displacements, elastic or inelastic energies, etc. According to some embodiments of the present disclosure, a first geometry can refer to the identification and characterization of unknown internal structures and defects with limited non-destructive measurements, and a second geometry can refer to designing geometry of solids to achieve optimized and/or improved mechanical performance including maximizing or minimizing individually or in certain combinations the stresses, strains, displacements, elastic or inelastic energies, etc.

The instant disclosure presents a unique, systematic approach based on PINNs for solving geometry identification and geometry design problems in continuum solid mechanics. PINNs integrate known PDEs of importance in solid mechanics with NNs, composing a unified computational framework involving both the forward solver and the inverse algorithm. Notably, the disclosure provides a method for directly parameterizing the geometry of the solid in a differentiable and trainable manner (i.e., geometry as trainable variables; geometry-dependent training points; making the gradient with respect to geometry tractable). According to the present disclosure, these geometric variables can be inputted into PINNs to advance the systems and methods described herein. An additional technique (i.e., pretraining procedure) is important for stabilizing the training process of the PINN. For identification problems, this method can concurrently solve unknown material parameters in addition to geometric parameters.

According to the exemplary embodiments described herein in detail, for identification problems, the PINN inversely characterizes the geometry of the void/inclusion according to the displacement data. For design problems, the PINN inversely design the optimized and/or improved geometry of the void/inclusion according to a predefined objective function. According to the present disclosure, this design process can also be referred to as generating the optimized geometry. According to some examples, optimized performance can refer to improving performance. As the method completes the identification/design task, it concurrently identifies the full field stresses, strains, displacements in the solid structure. This framework for inverse problems involving unknown (moving) domains directly parameterizes the computational domain (geometry), and can deal with material and geometry parameterization, even for large deformation.

To test the performance of the method with various parametric assessments, the instant disclosure builds a set of detailed cases for identification and design problems. The examples include different shapes and topologies of the structure and different constitutive models for describing the mechanical properties of the solid material, including linear elasticity, hyperelasticity, and deformation plasticity. For the particular case of inclusion in identification problems, the PINN is also required to estimate the unknown material parameter of the inclusion, through which the instant disclosure demonstrates the capability of our model in solving combined material and geometry identification problems. As a proof of concept for the method of the subject applications, the instant disclosure focuses on two-dimensional plane strain static problems. Result is accurate (relative error no more than O(10⁻²)).

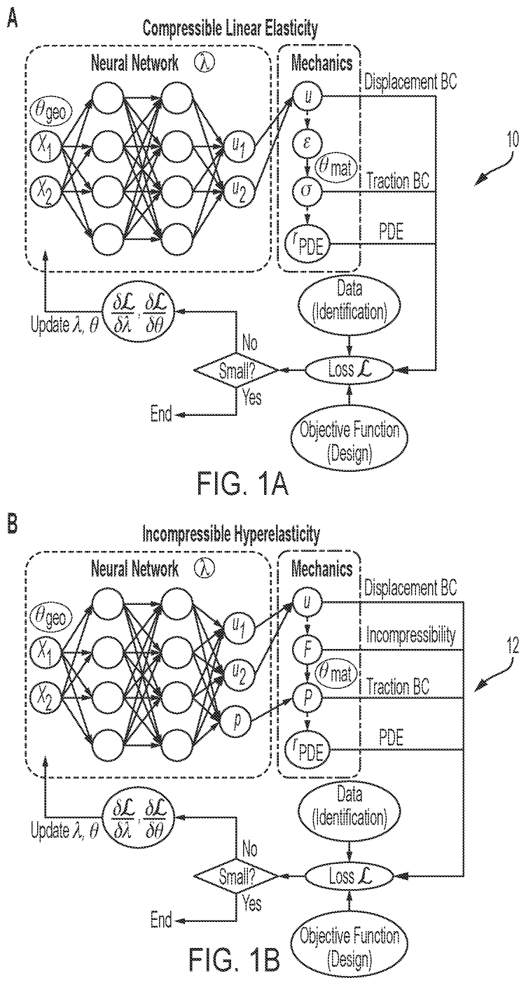

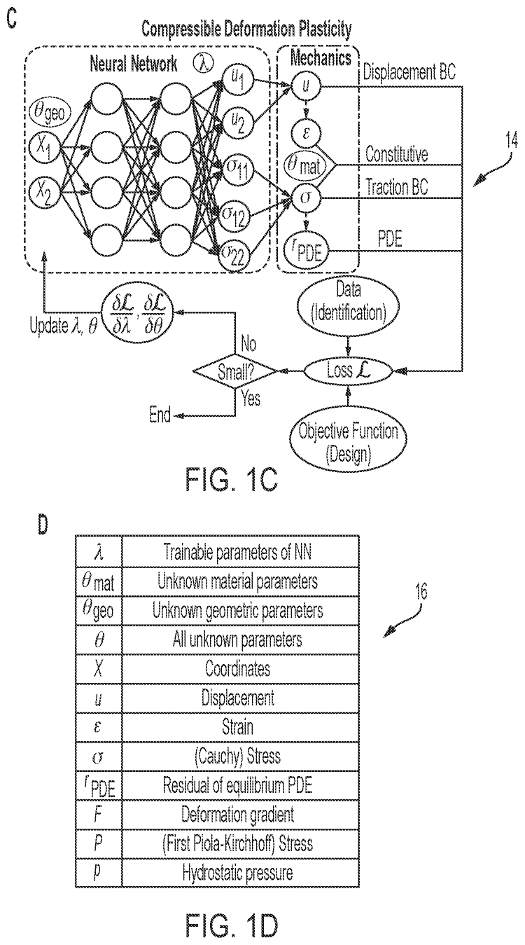

According to the exemplary embodiments described herein in detail, the present disclosure establishes the general formulation of PINNs in continuum solid mechanics involving both (material and geometry) identification and design problems. FIGS. 1A-1D present the architectures of PINNs for identification and design problems in continuum solid mechanics. Corresponding to computational examples of the present disclosure, design of the architectures of the PINNs for plane-strain problems for the three material models, as shown in view 10 of FIG. 1A for (compressible) linear elasticity, view 12 of FIG. 1B for (incompressible) hyperelasticity, and view 14 of FIG. 1C for (compressible) deformation plasticity is contemplated. The architectures of the PINNs are slightly different for different material models due to the characteristics of their mathematical expressions. For reference, view 16 of FIG. 1D includes the definitions of the mechanical quantities of interest in the architectures, which can apply to the interpretation of subsequent Figures of the present disclosure. Depending on the problems to solve (identification/design), the components of the loss function are also slightly different.

The present disclosure firstly summarizes the basic workflow of PINNs for identification problems as follows: First, it is contemplated that one can apply a neural network (NN; with trainable parameters λ) to approximate the primary solution fields (top left panels in FIG. 1A-1C) with respect to the in-plane coordinates X=(X₁, X₂). Secondly, it is contemplated that one can integrate the mechanical laws into the PINN architecture (top right panels in FIG. 1A-1C) by deriving relevant mechanical quantities of interest from the NN outputs, such as strain, stress, and the residual of equilibrium PDEs. In this process, unknown material parameters θₘₐₜ are involved. Thirdly, it is contemplated that one can formulate the loss function [symbol] (λ, θ), which measures the discrepancy between the predicted mechanical quantities of interest and their respective true values provided by mechanical laws and measured data (bottom right panels in FIGS. 1A-1C). For example, for linear elasticity in FIG. 1A, the loss function is expressed as

$$( λ , θ ) = {α}_{P D F} {P D E}_{} ( λ , θ ) + {α}_{BC} {BC}_{} ( λ , θ ) + {α}_{Data} {Data}_{} ( λ , θ ) , \qquad ( 1 )$$

where the α's with various subscripts refer to the weights of corresponding loss terms and the three loss terms correspond to PDEs, boundary conditions (BCs), and data, respectively. Each loss term [symbol] ⱼ(λ, θ) (where j=PDE, BC, Data) is the mean squared error evaluated on Nⱼ residual points

$${j}_{} ( λ , θ ) = \frac{1}{{N}_{j}} {\sum }_{i = 1}^{{N}_{j}} {\vert {r}_{j} ( {X}_{j}^{( j )} ( {θ}_{g e o} ) ; λ , {θ}_{mat} ) \vert}^{2} , \qquad ( 2 )$$

where rⱼ is the residual of the condition j at the ith residual point Xⱼ⁽ⁱ⁾(θ_{geo}). The Nⱼ residual points for condition j are distributed in the domain of condition j to correctly evaluate [symbol] ⱼ(λ, θ). The coordinates of the residual points Xⱼ⁽ⁱ⁾ depends on geometric parameters θ_{geo} because of the variable computational domain, which will be explained in detail in subsequent portions of the disclosure. Lastly, it is contemplated that one can conduct parameter estimation through the training of the PINN (bottom left panels in FIGS. 1A-1C), during which the unknown parameters θ=(θₘₐₜ, θ_{geo}) and neural networks parameters λ are updated/trained to minimize the loss function. This process can be expressed as:

$$\hat{λ} , \hat{θ} = {arg min}_{λ , θ} ( λ , θ ) , \qquad ( 3 )$$

where the hat symbol refers to the value of these trainable parameters after the training process completes. As the solution to the inverse problem, the estimation of the unknown parameters is \hat{θ}. More details of the formulation are shown throughout the remaining disclosure.

The aforementioned summary describes the procedure for solving geometry (and material) identification problems with the proposed methods. With minimum change of the loss function, this method can be applied to geometry design problems for optimized and/or improved performance, as also shown in FIGS. 1A-1C. According to the exemplary embodiments described herein in detail, the approach of parameterizing the geometry of computational domain can be applied to conducting structure design in fields of mechanical and civil engineering. For the PINN for linear elasticity, for instance, the loss function is modified as:

$$( λ , θ ) = {α}_{PDE} {PDE}_{} ( λ , θ ) + {α}_{BC} {BC}_{} ( λ , θ ) + {α}_{cnstr} {cnstr}_{} ( λ , θ ) + {α}_{target} {target}_{} ( λ , θ ) , \qquad ( 4 )$$

where the additional two loss terms [symbol] _{cnstr}(λ, θ) and [symbol] _{target}(λ, θ) correspond to the constraint in the design problem and the target function to be minimized in the problem. For example, in structural engineering, one often needs to design a structure with the maximum stiffness (hence minimum compliance) under a constraint for the total weight/volume of the structure. In this case, [symbol] _{cnstr}(λ, θ) penalize the violation of the weight/volume constraint, and [symbol] _{target}(λ, θ) may be defined as the compliance of the structure. In the training process, as the PINN seeks to minimize the loss function, the governing PDE and the boundary conditions are satisfied through minimizing [symbol] _{PDE}(λ, θ), [symbol] _{BC}(λ, θ); the constraints are approximately satisfied through minimizing [symbol] _{cnstr}(λ, θ); the design target is achieved through minimizing [symbol] _{target}(λ, θ). In this process, any relevant geometric parameters in θ are adjusted in order to minimize the loss function, thereby realizing the design of optimal geometry. Note that, in some situations, constraints can alternatively be directly incorporated through designing the architecture of PINN instead.

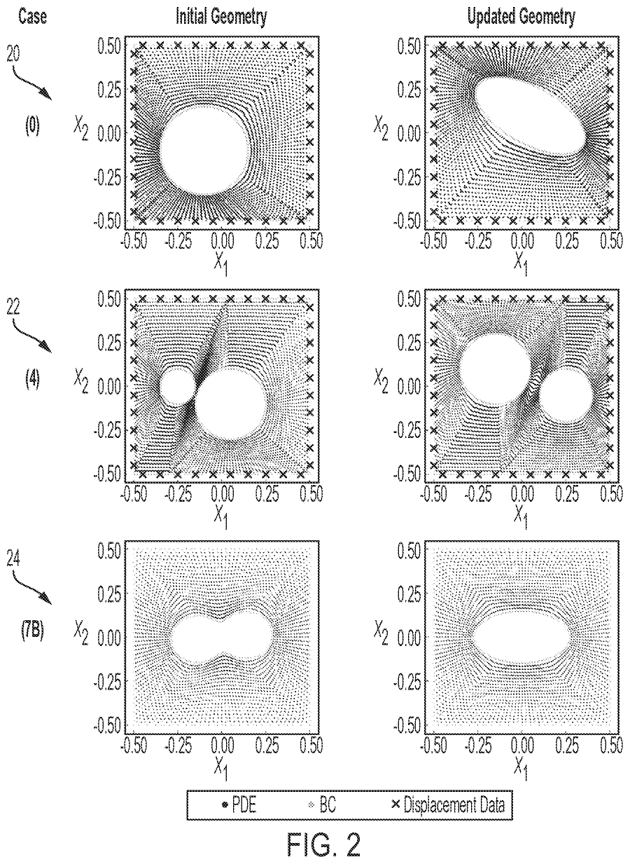

According to the exemplary embodiments described herein in detail, geometric parameters θ_{geo} play an essentially different role in the inverse problem compared to material parameters θₘₐₜ. Material parameters parameterize the governing PDEs of mechanics, which are naturally endowed with trainability through automatic differentiation of (physics-informed) neural networks. As a result, material parameters can be directly estimated using the standard formulation of PINNs for inverse problems. Geometric parameters θ_{geo}, on the other hand, parameterize the computational domains of the PDEs and boundary conditions, which do not naturally serve as trainable parameters in the framework of PINNs. To make the geometric parameters θ_{geo} differentiable and hence trainable in a similar way to material parameters θₘₐₜ, the present disclosure parameterizes the coordinates of residual points by geometric parameters θ_{geo}. Technically, it is contemplated that such parameterization can be implemented by utilizing the definition of trainable variables in deep learning libraries (e.g., TensorFlow, Pytorch): the present disclosure first defines the geometric parameters θ_{geo} as trainable variables; then, expresses the locations of residual points as functions of these trainable variables. As a result, the coordinates of residual points are automatically updated as the estimation of θ_{geo} are updated throughout the iterative training process. In FIG. 2, the present disclosure shows the residual points for each condition in Cases 0 and 4 of the examples for geometry identification at views 20 and 22, respectively, and Case 7B for geometry design at view 24, before and during the simulation. For reference, black dots are used to articulate PDE parametrization, and gray dots to articulate boundary conditions (BC). In views 20 and 24, displacement data is articulated by each bordering x″ character. In this way, the present disclosure ensures that the residual points for different conditions are always located in their correct domains. More importantly, this allows the present disclosure to capture the gradient of the loss function [symbol] with respect to the geometric parameters θ_{geo}, which otherwise could not be realized using the standard formulations of PINNs. With the geometry-parameterized residual points, the PINN can correctly update the estimated/designed geometric parameters θ_{geo} throughout the training process, thereby characterizing/designing the unknown geometry. It is contemplated that such form of parameterization invoking PINNs to solve geometry identification problems have hitherto not been addressed. Practically, θ_{geo} may be defined by assuming the shape of the void (e.g., circle, ellipse) and then assigning the unknown parameters to θ_{geo}; alternatively, for more flexibility, the void shape, or other shapes, may be represented by analytical function(s), parameterized function(s), non-uniform rational basis spline (NURBS) or another neural network, where a large number of parameters in θ_{geo} represents the irregular shape of the void. In this way, the shape of the at least one of one or more internal structures, internal surfaces, internal boundaries, external structures, external surfaces, or external boundaries can be arbitrarily complicated or simple (including but not limited to circular and elliptical shapes), or, as complicated as the methods of the present disclosure can describe.

In another aspect of the present disclosure, it is contemplated that one can find it important to pretrain the model before using the model to characterize/design unknown geometry. If the model is directly applied without pretraining, the estimated/designed geometric parameters rapidly depart from physically admissible values (e.g., void located outside the matrix) after a few iterations. Inspired by the transfer learning technique, the present disclosure proposes to maintain all the estimated unknown parameters θ fixed (not trainable) and only update the trainable parameters of the NN λ for the first few iterations. During this pretraining process, the PINN essentially solves a forward problem, seeking to roughly capture the qualitative pattern of the displacement field and the stress field. After this pretraining process, both λ and θ can be made trainable, which initiates the parameter estimation/design. Such a pretraining procedure induces λ to converge towards to the desired local minimum, hence serving as a good initialization for geometry identification and design problems. For the prototypical problem of the current disclosure, technically, the PINN needs to be pretrained until there emerged a qualitative pattern indicating the existence of a stress concentration around the curved boundary or interface.

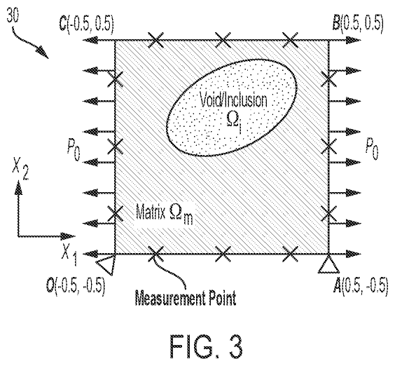

The present disclosure demonstrates the efficacy of the method for geometry identification problems by modeling a two-dimensional prototypical problem on a matrix-void/inclusion system as a proof of concept (see FIG. 3). A square-shaped matrix material contains a void/inclusion with unknown geometry. To characterize the location, size and shape of the void/inclusion, the instant disclosure applies loading P₀ on the matrix boundary and monitoring the displacement response on the measurement points at the matrix boundary under such loading.

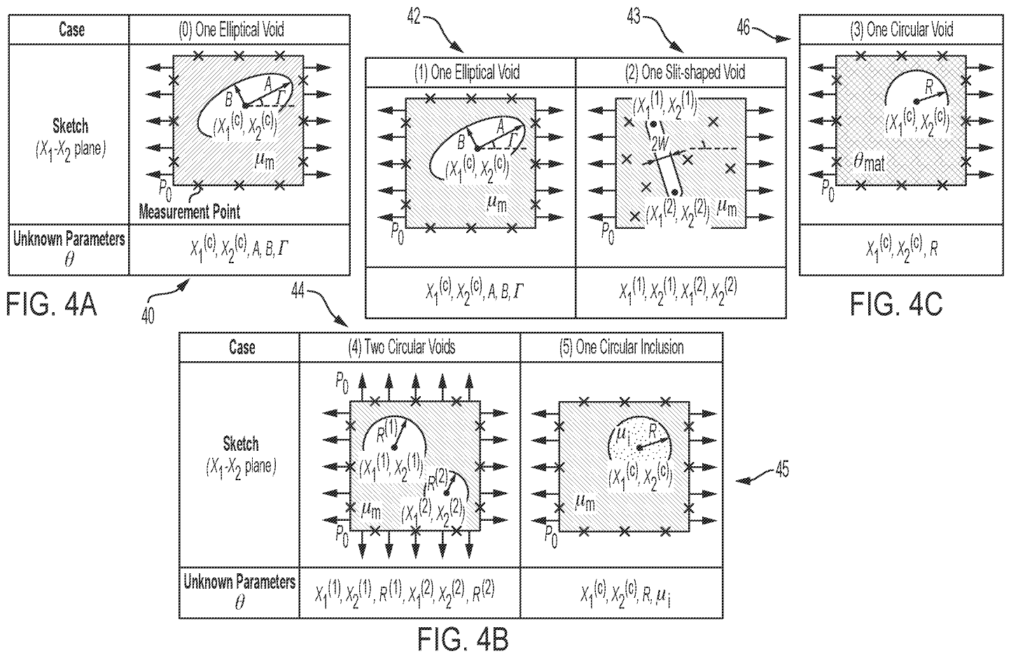

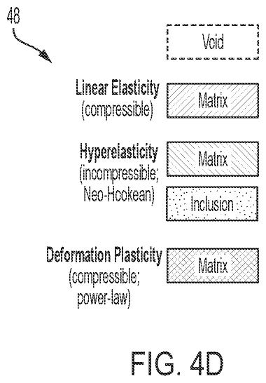

FIG. 3 includes the general setup of the prototypical problem on geometry and material identification in the present disclosure at view 30. In this way, FIG. 3 presents a plane-strain problem in the X₁-X₂ plane about a square-shaped matrix specimen with a void/inclusion. For example, the problem in FIG. 3 correlates to an incompressible Neo-Hookean hyperelasticity problem, and correlates to the sketching scheme presented in FIGS. 4A-4C. For the sketch of the X₁-X₂ plane, view 30 in FIG. 3 includes example measurement points at locations bordering the sketch marked “X”. The goal of the inverse problem is to estimate the geometric parameters θ_{geo} (and material properties θₘₐₜ) of the void (inclusion) Ωᵢ inside the matrix Ωₘ, by applying uniaxial/biaxial loading P₀ and collecting displacement data on the matrix boundary. For the instant disclosure, it is also contemplated that for the inclusion case, material properties of the inclusion are also characterized.

FIGS. 4A-4C present six specific plane-strain problems contemplated by the present disclosure, including the setup of Cases 0-5 of the prototypical problem. For reference, FIG. 4D provides a legend diagram 48 of the respective hashing schemes for FIGS. 3, 4A-4C, 6, and 8 relating to void, matrix, and inclusion problems of linear elasticity, hyperelasticity, and deformation plasticity. For each case, the instant disclosure specifies the type of the inhomogeneity (void/inclusion), the unknown parameters (θ_{geo} for void, or θ_{geo} and θₘₐₜ for inclusion; denoted together as θ=(θₘₐₜ, θ_{geo})), the material model (FIG. 4A, evaluating compressible linear elasticity at view 40; FIG. 4B, evaluating incompressible Neo-Hookean hyperelasticity at views 42, 43, 44, and 45; and FIG. 4C, evaluating compressible deformation plasticity at view 46), type of the loading (uniaxial/biaxial), and the location of displacement measurements (uniformly on the outer boundary/inside the solid). It is noted that for FIG. 4C, the sketch still refers to a X₁-X₂ plane, with unknown parameters also bordering the bottom of the diagram for inclusion. For each sketch of the X₁-X₂ plane, views 40-46 in FIGS. 4A-4C include example measurement points at locations bordering the sketch marked “X”. All unknown parameters describe the geometry of the void/inclusion except μᵢ in view 46 of FIG. 4C, for Case 5, which represents the shear modulus of the inclusion. The sketch and all the geometric parameters are shown in the reference (undeformed) configuration. It is contemplated that the material properties of the matrix are known quantities for all the cases.

It is noted that, in accordance with exemplary embodiments, the solution of the aforementioned Cases 0-5 in FIGS. 4A-4C will provide a proof of concept for the disclosed method under different practical scenarios, demonstrating the wide applicability of the method. The three material models (Cases 0, 1, and 3 in FIGS. 4A-4C as the baseline cases), cover a wide range of mechanical behavior patterns of natural and engineered materials in a vast array of practical applications. The present disclosure places the displacement measurement points only on the outer boundary of the matrix, to mimic the real-world situation where the internal details are not available, i.e., the situation where non-destructive measurements are required. Non-destructive measurements refer to measurement techniques for evaluating properties of materials, structures, and systems without causing damage or destruction, for which the feasibility of the proposed method will be illustrated by examples shown by Cases 0, 1, and 3-5. Case 2 explores the scenario of engineering application where the void has a large aspect ratio (such as a crack), which the present disclosure approximates by a slender slit. For this case only, the present disclosure allows the displacement measurements to be inside the solid due to the relative insensitivity of the boundary displacement with respect to the slit geometry. Case 4 demonstrates the applicability of the method for materials with multiple voids (such as porous materials or those with multiple cracks/slits). Case 5, estimates the material and geometric parameters for a soft circular inclusion to show that the method of the present disclosure can handle combined material and geometry identification problem.

Another aspect of the present disclosure adapts a finite element solver (e.g., commercial software such as Abaqus, open-source library such as FEniCS) to generate the computational examples. In this disclosure, Abaqus can be adopted as the finite element solver. Specifically, the present disclosure presets reference values of unknown parameters to be θ* and conducted forward simulations, which generated the displacement data provided to the PINN and ground-truth full-field solution for assessing the performance of the PINN. The PINN initialized the estimation of unknown parameters to be θ₀. The PINN firstly went through a pretraining procedure for stabilizing the forward prediction, where the estimated parameters were fixed to be θ*. As the PINN initiated parameter estimation through the iterative training process, it is contemplated that the estimated parameters θ would migrate towards the correct value θ*. The training process terminated after the loss function and the estimated parameters reached a relative plateau, yielding the parameter estimation results \hat{θ}. The detailed setups of the prototypical problem, the finite element solver, and the hyperparameters of the PINN for geometry identification problems are included in Paragraphs 00085-000105.

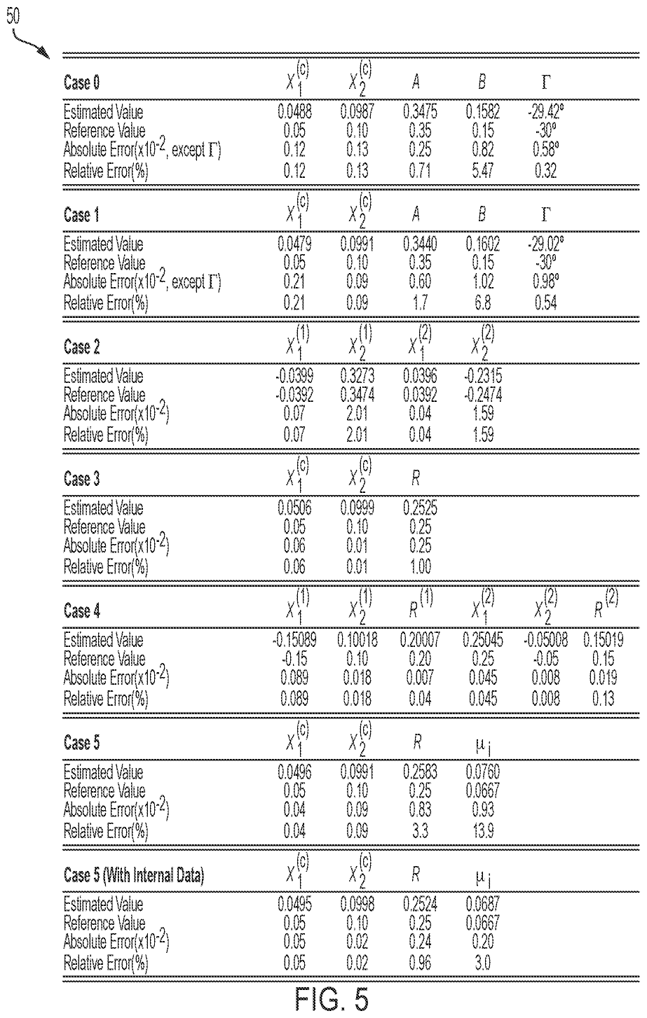

The present disclosure provides the results for Cases 0-5 in the main text. The results of parameter estimation for Cases 0-5 are shown in view 50 of FIG. 5. For each case, the present disclosure compares the estimated and reference values of the unknown parameters by presenting absolute errors and relative errors. To calculate the relative error, the present disclosure normalizes the coordinates, the lengths and the modulus, and the tilting angle by the domain size (side length of the matrix), respective reference values, and 180°, respectively. FIG. 5 indicates that the PINN estimates unknown parameters with high accuracy, with relative error O(10⁻²) on most parameters and as small as O(10⁻⁴) for some parameters.

It is worth noting that the estimated shear modulus of the inclusion μᵢ in Case 5 has an error slightly more than 10%. To improve the accuracy of Case 5, the present disclosure supposes that five additional data points inside the solid are available as in Case 2. The present disclosure re-trains the PINN with the expanded measurement data and append the results as the modified Case 5, labeled Case 5 (With Internal Data) in FIG. 5. With the additional data, the relative error of estimated parameters decreases to O(10⁻²), similar to other cases. In summary, given scattered displacement measurements, the PINN can accurately characterize the geometry (and material properties) of the internal void(s)/inclusion for various problem setups, including different constitutive relations, shapes of voids, and numbers of voids. The result indicates the generality of the method of the present disclosure for solving a broad spectrum of inverse problems in mechanics of materials.

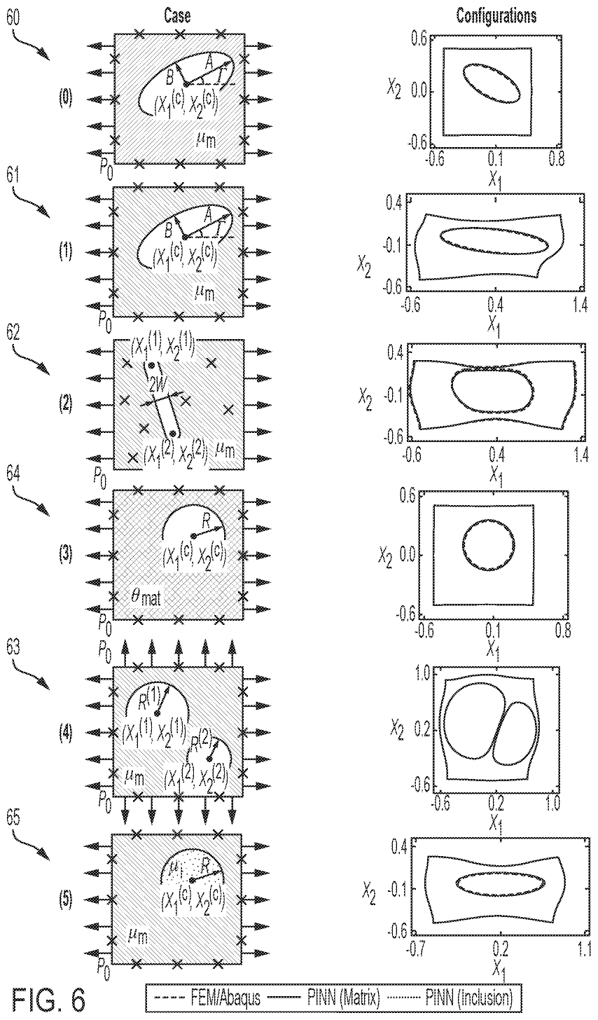

It is noted that, in accordance with exemplary embodiments, the method of the present disclosure is not only capable of estimating unknown parameters, but also providing quantitative measures of the deformed patterns of the solid as well. Specifically, the present disclosure applies the estimation results \hat{λ} and \hat{θ}_{geo} (see Eq. 3) on the neural network part of the PINN (top left panels in FIGS. 1A-1C) to infer the deformed configuration, where \hat{θ}_{geo} determines the reference (undeformed) configuration, and \hat{λ} determines the mapping from the reference (undeformed) configuration to the deformed configuration. In FIG. 6 (views 60-65), the present disclosure displays the comparison of the deformed configurations between the FEM ground truth and the PINN inference results for Cases 0-5. Again, FIG. 6 includes example measurement points at locations bordering the sketch marked “X” across Cases 0-5. For clarity of presentation, FIG. 6 shows the outer and inner boundaries of the specimen visualized from the FEM and PINN analyses in the accompanying “Configurations” for Cases 0-5. For each of the resulting Configurations, the dashed lines correlate with FEM/Abaqus, the straight black line with PINN (Matrix), and the dots with PINN (Inclusion) metrics. The two outlines match each another to a high extent, indicating that the deformed configurations from the PINN are almost identical to those from the FEM ground truth. For Case 5 specifically, the inner boundary of the matrix and the boundary of the inclusion predicted by the PINN also overlap well with each other, indicating that the continuity of the material surfaces in the matrix-inclusion system is preserved in the inference of the PINN.

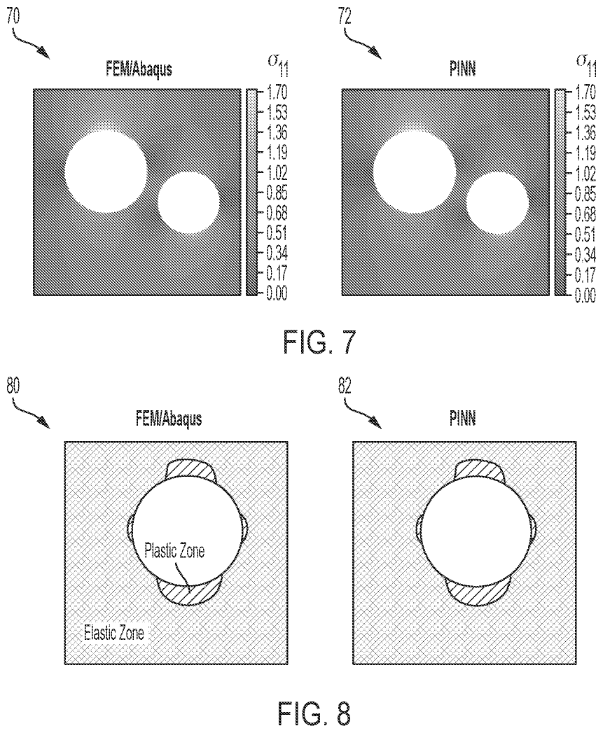

In addition to the reconstruction of deformation pattern, the method of the present disclosure is also capable of reconstructing the distribution of related mechanical quantities within the entire volume of the solid, including strain and stress components, principal stresses, elastic energy density, etc. For Case 4, for example, can be shown in FIG. 7 a comparison of the normal (Cauchy) stress in the horizontal direction (σ₁₁) from the PINN prediction at view 72 and the FEM reference solution at view 70, which demonstrates that our method is able to reconstruct the distribution of σ₁₁. Similarly, other mechanical variables can be obtained and reconstructed. For Case 3, where plasticity is involved, the present disclosure also examines the inference of the plastic zone. FIG. 8, also using legend 48 from FIG. 4D relating to deformation plasticity, shows the comparison of the plastic zone between the PINN prediction at view 82 and the FEM ground truth at view 80. Not only is the geometry of the void characterized correctly as previously verified in FIG. 5 and FIG. 6, the plastic zone of the loaded matrix is also inferred with high accuracy.

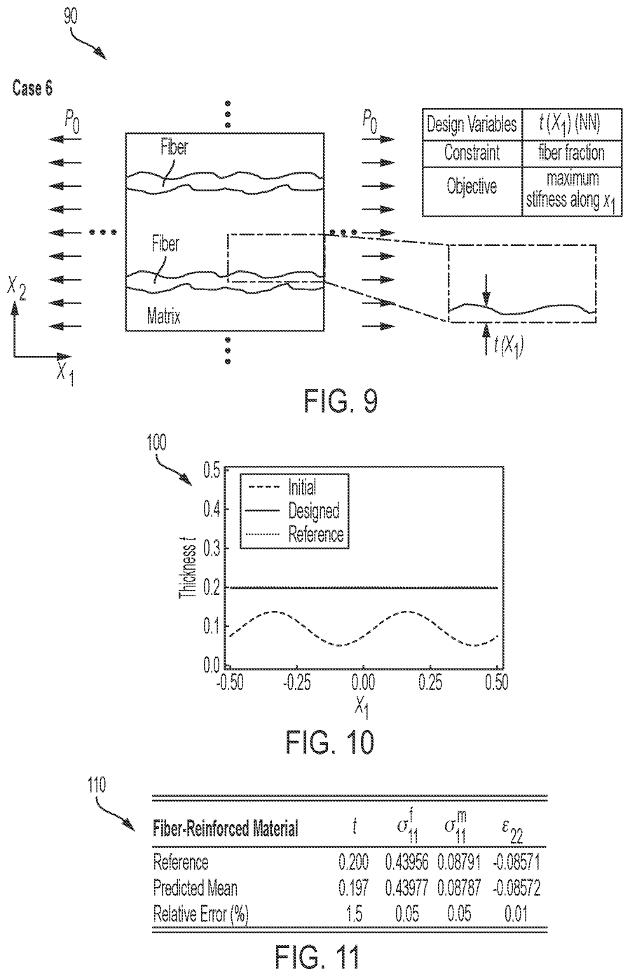

In addition to the aforementioned Cases 0-5 on inverse material/geometry identification problems, the present disclosure also considers illustrative examples of inverse problems on the design of optimal geometry. As a first illustrative example, this disclosure considers a problem on designing fiber-reinforced materials (called as Case 6 hereinafter), as shown in diagram 90 of FIG. 9. The composite material is composed of homogeneous matrix (relatively soft, governed by linear elasticity with Young's modulus Eₘ=0.4 and Poisson's ratio v=0.3) and periodically, horizontally aligned fibers (relatively stiff, governed by linear elasticity with Young's modulus E_{f}=2.0 and Poisson's ratio v=0.3). The target of Case 6 is: given the constraint on the maximum area fraction of the fiber (20% in our example), design the shape of the fiber so that the material achieves maximum stiffness under horizontal uniaxial loading (i.e., minimum mean normal stress in x₁ direction when applied with a fixed strain loading).

To solve this problem, the subject disclosure proposes to represent the fiber-matrix interface with a NN which takes x₁ (the horizontal coordinate) as input and outputs/(the thickness of the fiber at the given x₁). The trainable parameters of this NN are viewed as unknown geometric parameters to be designed. A representative volume element (the dashed box in FIG. 9) with size 1.0×1.0 is selected for the mechanical analysis. Similar to the inverse identification problem, the residual points are adaptively assigned according to the geometry of the interface. [symbol] _{cnstr} is defined to make sure that fiber makes up 20% of the area/volume. Uniform strain loading is applied on left and right boundaries. [symbol] _{target} is defined as the mean normal stress in the x₁ direction on residual points. As the shape of the interface changes, the present disclosure anticipates that this mean normal stress response is maximized, which corresponds to the optimal design with maximum stiffness. Theoretical derivations for composite materials have revealed that the maximum stiffness is achieved under the isostrain state, which means that the thickness of the fiber can be uniform (in our case t(x₁)=0.2 for all x₁) for achieving maximum stiffness in x₁ direction.

Continuing to graph 100 in FIG. 10, which details the results of Case 6, where the interface profile t(x₁) is shown for the random initialization before the design process and after the design process. After the iterative design process, the thickness profile becomes uniform and converges to be close to t(x₁)=0.2 for all x₁∈(−0.5, 0.5), which matches the theoretical optimal design. The quantitative comparison of the design results to theoretical optimum is shown in view 110 of FIG. 11. The results indicate that our method based on PINNs have successfully identified the optimal design for of the fiber-reinforced material in Case 6 a proof of concept.

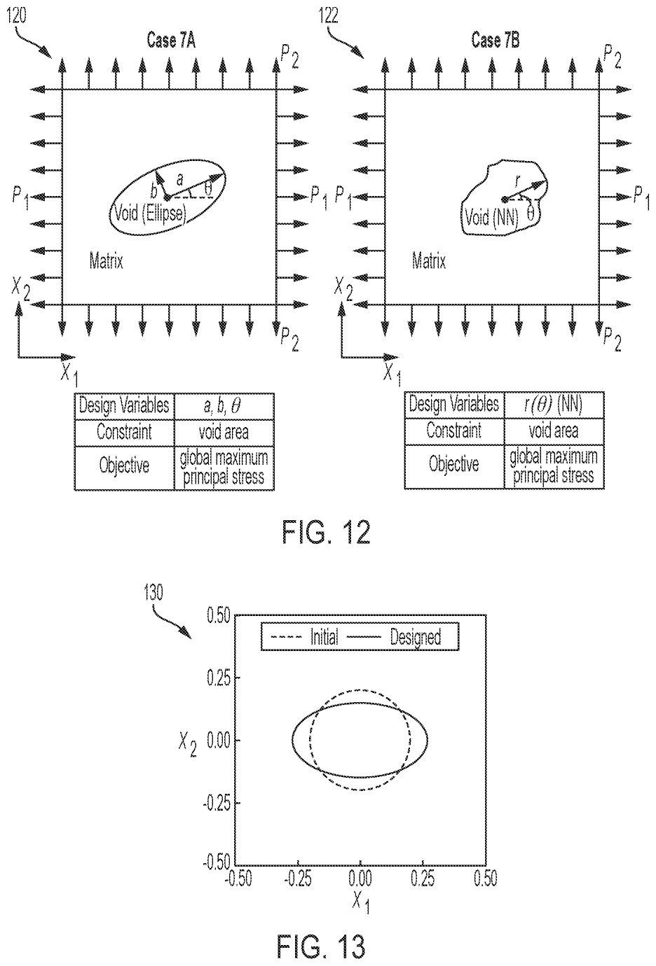

An additional illustrative example (Case 7) is presented in views 120 and 122 respectively of FIG. 12, which presents a matrix-void system. This aspect of the disclosure is similar to the Cases 0-4 for the inverse identification problem (see FIG. 3). The matrix is made of linear elastic material with Young's modulus E=1.0 and Poisson's ratio v=0.3. The target of the problem is to minimize the global maximum principal tensile stress in the entire domain under the uniform, unequal biaxial loading (with magnitude P₁=0.6 in x₁ direction and P₂=0.3 in x₂ direction) provided that the area of the void is fixed (equal to the area of a circle with radius r₀=0.2).

The instant disclosure considers two situations regarding the geometry parameterization: (1) the void is constrained to be a centered ellipse (Case 7A in FIG. 12, shown in view 120), so that it has only three design variables, including semi-major axis a, semi-minor axis b (which essentially compose one independent design variable a/b due to the area constraint of the void) and the tilting angle of the elliptical void (θ); (2) the void can be any shape which is described by a NN representing the void boundary in polar coordinates r(θ) centered at the matrix center (Case 7B in FIG. 12, shown in view 122). To describe the variable geometry, the instant disclosure defines the unknown geometric parameters as trainable variables for Case 7A, and apply a neural network to represent the void boundary in polar coordinates for Case 7B. In Case 7B, the trainable variables of the NN representing r(θ) can be viewed as unknown geometric parameters. Case 7A is compared with finite element simulations to demonstrate the optimality of our design. For Case 7B, on the other hand, the present disclosure shows the proof of concept of our method in designing an arbitrary geometry.

Notably, in this example, the instant disclosure enforces the constraint of the area not through the addition of loss term [symbol] _{cnstr} as a soft constraint. Instead, the constraint is realized in a hard way by normalizing the void geometry following the output of the NN for describing r(θ), so that the area is fixed whatever the trainable parameters are.

This aspect of the instant disclosure examines the results in Case 7A from the PINN for designing the aspect ratio a/b and the tilting angle θ to minimize the maximum value of the maximum principal tensile stress in the entire solid. Due to the setup of the biaxial loading with P₁:P₂=2:1, if the void is circular, the largest tensile stress will be located at the top/bottom of the void. To avoid this, the void is anticipated to increase its aspect ratio and align its major axis along x₁ direction, so that a less sharp (a larger curvature radius) at the top/bottom of the elliptical void reduces the local stress concentration.

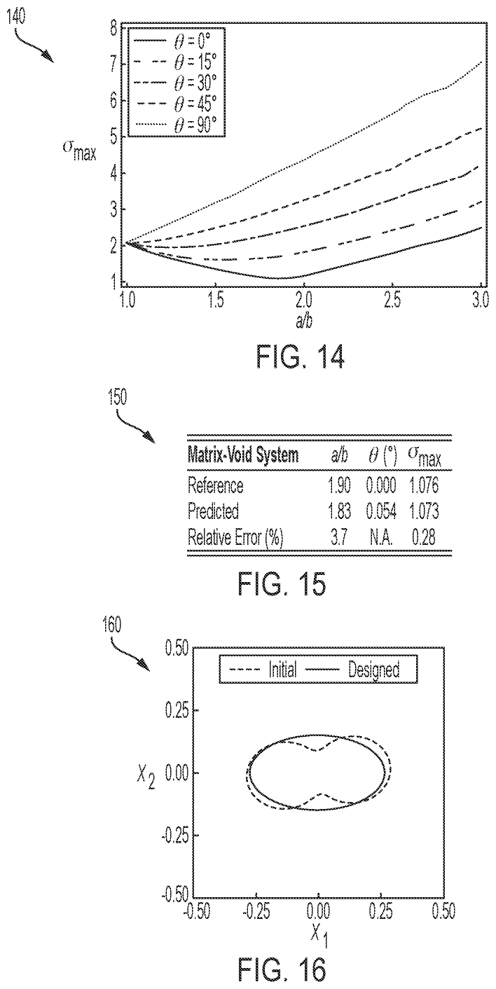

The results of the void outline for Case 7A are shown in graph 130 of FIG. 13, with dashed lines correlating to the initial shape and solid lines correlating with the designed shape. After the shape initialization and the beginning of the design process, the shape of the void evolves as anticipated: the major axis aligns along x₁ direction, and the aspect ratio is roughly 2:1 to match P₁:P₂. To quantitatively verify the correctness of our design, the maximum principal tensile stress can be computed for various values of the aspect ratio a/b and the tilting angle θ of the void with finite element method which serves as the reference solution. The maximum principal tensile stress σₘₐₓ for different a/b and θ is shown in graph 140 of FIG. 14. In FIG. 14, the respective line dashing schemes correlate with the respective tilting angle θ of the void. The quantitative comparison of the final design results is shown in view 150 of FIG. 15. The results indicate that our method based on PINNs have identified the optimal design with high accuracy.

Graph 160 of FIG. 16 presents the results of the void outline for Case 7B, with dashed lines correlating to the initial shape and solid lines correlating with the designed shape. This aspect of the instant disclosure tests the performance of our PINN model in designing the void where arbitrary shape is admissible through the introduction of a NN for describing the shape of the boundary. Although the setup of the initial void is to be a dumbbell shape, the shape of the void still evolves to converge to an ellipse-like shape as the design process is performed.

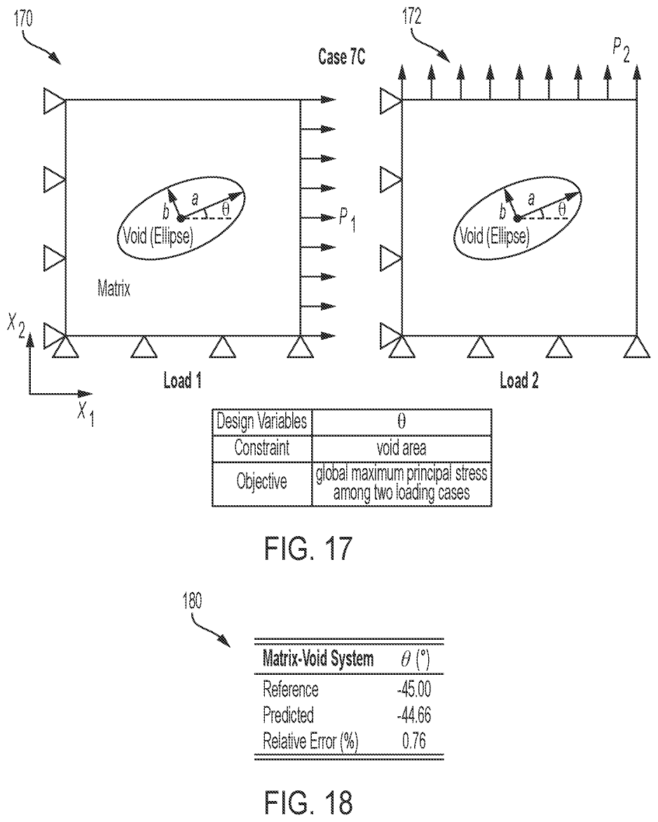

In addition to the aforementioned Cases 7A and 7B for the matrix-void system, the present disclosure also examines the method performance in terms of more complicated problems involving multiple mechanical loading conditions. FIG. 17 presents an illustration of Case 7C. The matrix in this case is assumed to be hyperelastic, which undergoes large deformation under loading. The present disclosure considers two different loading cases simultaneously at 170 and 172, respectively (see FIG. 17), in which the matrix is uniaxially stretched in x₁ and x₂ directions for the two loading conditions, respectively. The goal of this problem is to minimize the maximum value of the global maximum principal tensile stress among the two loading conditions. The void is fixed to be an ellipse with lengths of the two semi axes being a=0.35 and b=0.15. The only unknown parameter of the geometry is the tilting angle θ. The optimal tilting angle for minimizing this maximum principal tensile stress is ±45° according to symmetry. View 180 of FIG. 18 shows comparison of the final value after the training completes, which demonstrates that the method in the disclosure has well designed the optimal geometry for minimizing the global maximum principal tensile stress.

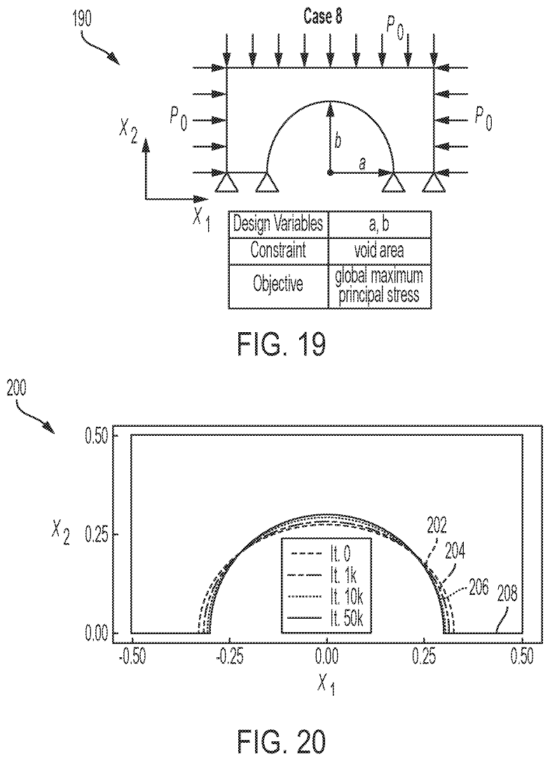

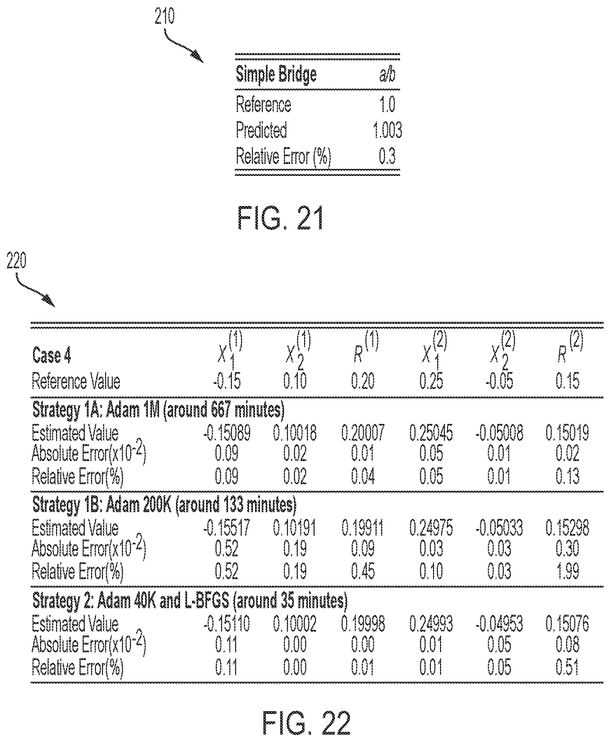

The instant disclosure considers an example (Case 8) of designing the shape of a simple bridge as a proof of concept for demonstrating the practical applicability of our method in mechanical and civil engineering as shown in diagram 190 of FIG. 19. This illustrative embodiment considers a bridge (with size 1.0×0.5; the structure is governed by linear elasticity with Young's modulus E=1.0, Poisson's ratio v=0.3) under compressive loading (with pressure P₀=0.2) from top, left and right directions. The bottom edge is fixed in x₂ direction. The present disclosure further assumes that the shape of the bridge arc is a semi-ellipse with one axis overlapping with the base of the structure. Given the constraint on the total volume of the bridge (=71.73% of the rectangular region 1.0×0.5), the target of the instant disclosure is to design the optimal aspect ratio (a/b, where a and b are the lengths of the two axes of the ellipse) of the elliptical arc such that the global maximum principal tensile stress is minimized. Due to the equal biaxial compressive loading P₀, the optimal shape can be a semi-circle. Results are shown in view 200 of FIG. 20 and view 210 of FIG. 21, respectively, with dashed lines correlating to the initial shape and solid lines correlating with the designed shape. In view 200 of FIG. 20, 202 refers to 0 iterations, 204 to 1,000 iterations, 206 to 10,000 iterations, and 208 to 50,000 iterations, respectively. After the design process is completed, the arc deviates from its initial shape and finally becomes a semi-circle. The aspect ratio of the two axes of the semi-elliptical arc after design is 1.003, which has a relative error as small as 0.3% compared to the analytical solution of 1.0.

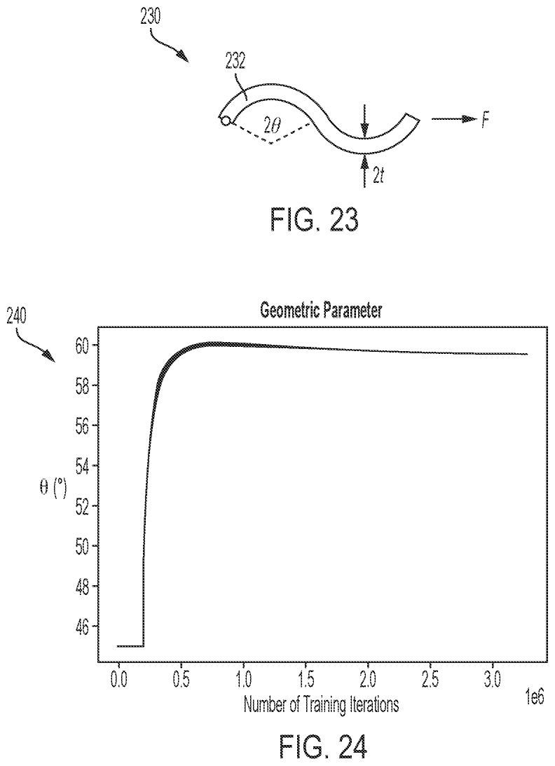

The present disclosure includes an additional computational case considering multiple loading conditions in addition to the aforementioned Case 7C. The illustrative example (Case 9) considers applying multiple loadings on a structure and seeking to optimize the performance of the structure under a metric that involves these loading conditions. Technically, to implement such problems, it is noted that the PINN is slightly modified to include an additional pseudo-time/as the input of the neural network, which essentially indicates the loading steps. As a proof of concept, the illustrative example of the present disclosure considers a curved beam structure 232, as shown in view 230 of FIG. 23. The distance between the two ends is fixed to be 1.0. The beam 232 shown in view 230 of FIG. 23 consists of two identical arcs with width 2t, each with a curved angle being 2θ. On the two ends of the beam 232, relative displacement in the horizontal direction (ū=0.0, 0.1, 0.2, . . . , 1.0) is applied. According to the illustrative example, periodic boundary conditions are applied to ensure that the cross sections on the ends are parallel. The target of the design problem focuses on the slope of the tangent of the force-displacement curve. Thereby, the illustrative example denotes the force response under the enforced displacement ū as F(ū). The design optimization problem is defined to adjust the curved angle θ, so that:

$$\frac{F ( 0.3 ) - F ( 0 )}{0.3 - 0.} \approx 0.0035 \qquad ( 2 )$$

$$\frac{F ( 0.9 ) - R ( 0.8 )}{0.9 - 0.8} \approx 0.0045 . \qquad ( 3 )$$

The illustrative example of the present disclosure seeks to find a target value θ through these two design targets, so that the homogenized (equivalent) stiffness of the curved beam 232 satisfies the desired values under both small and large deformations.

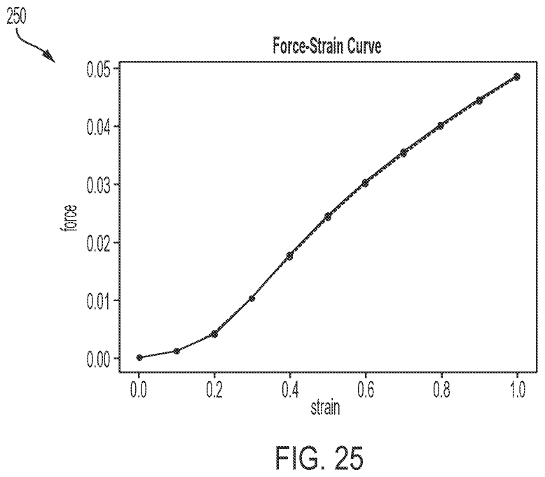

The results are shown in graphs 240 and 250 of FIGS. 24 and 25, respectively. The geometric parameter θ is initialized to be 45°. The target slopes of the problem are chosen such that θ=60° provides the design closest to the target. According to FIG. 24, the designed parameter deviates from the initial value 45° and approaches 60°, as shown in graph 240 detailing the evolution of the designed geometric parameter θ. FIG. 25 shows the comparison of the force-strain curve between the designed geometry predicted by the PINN (solid line) and the ground-truth curve corresponding to θ=60° (dashed line) in graph 250. The results presented in the illustrative example demonstrate that the PINN, together with the proposed approach for the parameterization of geometric parameters, is capable of designing optimal geometry under customized targets. In particular, this case demonstrates the method's feasibility for complicated problems involving multiple boundary-value problems with different loading conditions.

This aspect of the present disclosure further provides the detailed formulation of PINNs for forward and inverse problems in continuum solid mechanics. This focus concerns the PINN for hyperelasticity (specifically, incompressible Neo-Hookean material) as most of the computational examples of the present disclosure adopt this material. To better clarify the quantitative formulation, here the present disclosure denotes all the material and geometric parameters of interest as θₘₐₜ and θ_{geo}, respectively. For incompressible Neo-Hookean materials, the only material parameter is the shear modulus μ so that θₘₐₜ=μ. The unknown part of θ=(θₘₐₜ, θ_{geo}) in the inverse problem is denoted as θᵤₙₖ.

The workflow of PINNs comprises four steps in the present disclosure. First, it is contemplated that one can apply a NN to approximate the primary solution fields (top left panel in FIG. 1B) in domain Ω(θ_{geo}), including the displacement field u(X; λ) and the pressure field p(X; λ), where λ represents trainable parameters of the NN, X=(X₁, X₂) is the in-plane coordinates in the reference/undeformed configuration, and the quantities with tilde represent the approximation from the neural network. For incompressible materials, the present disclosure envisions the hydrostatic pressure field p as a Lagrange multiplier accompanying the displacement field u to uniquely determine the stress field.

Secondly, it is contemplated that one can integrate mechanical laws into the PINN architecture (top right panel in FIG. 1B) by deriving relevant mechanical quantities of interest from the NN outputs. During this calculation process, partial derivatives are handled by automatic differentiation. The deformation gradient F(X; λ) and the first Piola-Kirchhoff stress P(X; λ, μ) are calculated by:

$$\tilde{F} ( X ; λ ) = I + \frac{\partial \tilde{u}}{\partial X} ( X ; λ ) , \qquad ( A1 )$$

$$\tilde{P} ( X ; λ , μ ) = - \tilde{p} ( X ; λ ) {\tilde{F}}^{- 1} ( X ; λ ) + μ \tilde{F} ( X ; λ ) , \qquad ( A2 )$$

where I is the identity tensor, Eq. A1 is kinematics, and Eq. A2 is the constitutive relation for incompressible Neo-Hookean materials. The residuals of the equilibrium PDE and the incompressibility condition at X are expressed by:

$${\tilde{r}}_{PDE} ( X ; λ , μ ) = Div \tilde{P} ( X ; λ , μ ) , X ∈ Ω ( {θ}_{g e o} ) , \qquad ( A3 )$$

$${\tilde{r}}_{inc} ( X ; λ ) = det ( \tilde{F} ( X ; λ ) ) - 1 , X ∈ Ω ( {θ}_{geo} ) . \qquad ( A4 )$$

The residuals of Dirichlet/displacement and Neumann/traction boundary conditions at X are:

$${\tilde{r}}_{D} ( X ; λ ) = \tilde{u} ( X ; λ ) - \overline{u} ( X ) , X ∈ {ψΩ}_{D} ( {θ}_{geo} ) , \qquad ( A5 )$$

$${\tilde{r}}_{N} ( X ; λ , μ ) = \tilde{P} ( X ; λ , μ ) N ( X ) - \overline{T} ( X ) , X ∈ \partial {Ω}_{N} ( {θ}_{geo} ) , \qquad ( A6 )$$

where N is the outward unit normal vector on the boundary, and u and T are the specified displacement and traction on the boundary, respectively. For inverse problems, the present disclosure provides displacement data{u*⁽ⁱ⁾}ᵢ₌₁^{N}^{ᵤ} at {Xᵤ⁽ⁱ⁾}ᵢ₌₁^{N}^{ᵤ}. The residual of the ith displacement observation is:

$${\tilde{r}}_{u}^{( i )} ( λ ) = \tilde{u} ( {X}_{u}^{( i )} ; λ ) - {u}^{* ( i )} . \qquad ( A7 )$$

Thirdly, it is contemplated that one can formulate the loss function according to the foregoing residuals from mechanics and data (bottom right panel in FIG. 1B). To define the loss terms corresponding to the problem definition, the present disclosure places N_{Ω}, N_{D} and N_{N} residual points in Ω, on ∂Ω_{D} and ∂Ω_{N}, denoted as X_{Ω}⁽ⁱ⁾ (i∈{1,2, . . . , N_{Ω}}), X_{D}⁽ⁱ⁾ (i∈{1,2, . . . , N_{D}}), X_{N}⁽ⁱ⁾ (i∈{1,2, . . . , N_{N}}), respectively. Since the present disclosure parameterizes the coordinates of residual points by θ_{geo}, these residual points are all parameterized by θ_{geo}. Then, it is contemplated that one can evaluate the mean squared residuals of the PDEs, Dirichlet and Neumann boundary conditions, respectively. Each loss term is defined by

$${PDE}_{} ( λ , θ ) = \frac{1}{{N}_{Ω}} {\sum }_{i = 1}^{{N}_{Ω}} {\vert {\tilde{r}}_{PDE} ( {X}_{Ω}^{( i )} ( {θ}_{geo} ) ; λ , μ ) \vert}^{2} \qquad ( A8 )$$

$${inc}_{} ( λ , θ ) = \frac{1}{{N}_{Ω}} {\sum }_{i = 1}^{{N}_{Ω}} {\vert {\tilde{r}}_{inc} ( {X}_{Ω}^{( i )} ( {θ}_{geo} ) ; λ ) \vert}^{2} \qquad ( A9 )$$

$${D}_{} ( λ , θ ) = \frac{1}{{N}_{D}} {\sum }_{i = 1}^{{N}_{D}} {\vert {\tilde{r}}_{D} ( {X}_{D}^{( i )} ( {θ}_{geo} ) ; λ ) \vert}^{2} \qquad ( A10 )$$

$${N}_{} ( λ , θ ) = \frac{1}{{N}_{N}} {\sum }_{i = 1}^{{N}_{N}} {\vert {\tilde{r}}_{N} ( {X}_{N}^{( i )} ( {θ}_{geo} ) ; λ , μ ) \vert}^{2} \qquad ( A11 )$$

$${u}_{} ( λ , θ ) = \frac{1}{{N}_{u}} {\sum }_{i = 1}^{{N}_{u}} {\vert {\tilde{r}}_{u}^{( i )} ( λ ) \vert}^{2} , \qquad ( A12 )$$

and the loss function is:

$$( λ , θ ) = {α}_{PDE} {PDE}_{} ( λ , θ ) + {α}_{inc} {inc}_{} ( λ , θ ) + {α}_{D} {D}_{} ( λ , θ ) + {α}_{N} {N}_{} ( λ , θ ) + {α}_{u} {u}_{} ( λ , θ ) , \qquad ( A13 )$$

where α_{PDE}, α_{inc}, α_{D}, α_{N}, αᵤ are the weights of the loss terms. Note that the two loss terms [symbol] _{D} and [symbol] _{N} for the two types of boundary conditions are simplified into [symbol] _{BC} in Eq. 1 in the main text.

Lastly, it is contemplated that one can conduct parameter estimation through training/loss minimization (bottom left panel in FIG. 1B). The trainable parameters of the PINN include the trainable parameters of the NN, λ, and the unknown parameters of the inverse problem, θᵤₙₖ(∈θ). Using the notations in this section, this process can be expressed as:

$$\hat{λ} , {\hat{θ}}_{unk} = {arg min}_{λ , {θ}_{unk} ⊆ θ} ( λ , θ ) . \qquad ( A14 )$$

With the PINN adjusting λ to minimize the loss function, it is contemplated that one can anticipate that all the mechanical laws will be approximately satisfied, making the NN serve as an approximation to the primary solution fields. Furthermore, the residual of displacement observations in the loss function guides the estimated unknown parameters to evolve towards their respective target values. In this way, the PINN is able to solve inverse problems.

The present disclosure further contemplates the differences of PINNs for linear elasticity (FIG. 1A) and deformation plasticity (FIG. 1C). For these two constitutive relations, the solid undergoes infinitesimal deformation, so that one does not need to distinguish reference (undeformed) configuration (X₁, X₂) and deformed configuration (x₁, x₂). In this section, the coordinates are written as (x₁, x₂) to keep consistent with the conventional notations in solid mechanics community. Due to the compressibility, the hydrostatic pressure p is no longer an independent state variable, so that p is not needed as a primary output of the NN.

To build the PINN for linear elasticity, kinematics in Eq. A1 is replaced by the definition of the infinitesimal strain tensor εᵢⱼ=∂uᵢ/∂xⱼ, where u=(u₁, u₂) is the displacement. Stress-strain relationship in Eq. A2 is replaced by the stress-strain relationship

$${σ}_{ij} = {λε}_{kk} {δ}_{ij} + 2 {με}_{ij} , \qquad ( A15 )$$

where λ and μ are Lamé constants. The equilibrium equation similar to Eq. A3 is ∂σᵢⱼ/∂xⱼ=0. Note that the loss function for (compressible) linear elasticity does not require the term for incompressibility (see Eq. 1 and Eq. A13).

The PINN for the power-law deformation plasticity can be constructed based on the PINN for linear elasticity. In this case, the nonlinear stress-strain relation is expressed as

$$E ε = σ + \frac{3}{2} {α ( \frac{{σ}_{c}}{{σ}_{Y}} )}^{n - 1} s , \qquad ( A16 )$$

where E (Young's modulus), α, α_{Y} (yield stress), and n (hardening exponent) are material parameters. s is the deviatoric stress tensor

$$( {s}_{ij} = {σ}_{ij} - \frac{1}{3} {σ}_{kk} {δ}_{ij} ) , and {σ}_{e} ( = \sqrt{\frac{3}{2} {s}_{ij} {s}_{ij}} )$$

is the von Mises stress.

According to Eq. A16, stress cannot be explicitly expressed by strain for power-law deformation plasticity, unlike linear elasticity (FIG. 1A) and hyperelasticity (FIG. 1B). Therefore, the integration of constitutive relation through the analytical expression of stress tensor no longer works for deformation plasticity. As an alternative solution, the present disclosure includes the stress field σ=(σ₁₁, σ₁₂, σ₂₂) as primary solution field of the neural network in addition to the displacement field u=(u₁, u₂) (see FIG. 1C). After calculating the strain field according to Eq. A15, one can bridge the strain and stress fields with an additional loss term according to Eq. A16. In this way, the present disclosure integrates constitutive relation as an additional penalty term in the loss function for deformation plasticity.

According to the exemplary embodiments herein, in the case of inverse problems for multiple materials as in Case 5 in the main text, the present disclosure applies two independent NNs to approximate the displacement and pressure fields of the matrix and inclusion materials, respectively. Such approximation can be expressed by

$$Matrix : ( \tilde{u} ( X ; {λ}_{m} ) , \tilde{p} ( X ; {λ}_{m} ) ) , X ∈ {Ω}_{m} ( {θ}_{geo} ) \qquad ( A17 )$$

$$Inclusion : ( \tilde{u} ( X ; {λ}_{i} ) , \tilde{p} ( X ; {λ}_{i} ) ) , X ∈ {Ω}_{i} ( {θ}_{geo} ) \qquad ( A18 )$$

In addition to the foregoing loss terms for each material, an additional loss term [symbol] ᵢₙₜ can be utilized to force the continuity of displacement and traction on the interface Γᵢₙₜ(θ_{geo}) of two materials. To do this, the residuals can be written first on a single point for the two conditions of continuity. The residual of displacement continuity is

$${\tilde{r}}_{Dint} ( X ; {λ}_{m} , {λ}_{i} ) = \tilde{u} ( X ; {λ}_{m} ) - \tilde{u} ( X ; {λ}_{i} ) , X ∈ {Γ}_{int} ( {θ}_{geo} ) , \qquad ( A19 )$$

and the residual of stress continuity is

$${\tilde{r}}_{Nint} ( X ; {λ}_{m} , {λ}_{i} , {μ}_{m} , {μ}_{i} ) = \tilde{P} ( X ; {λ}_{m} , {μ}_{m} ) N ( X ) - \tilde{P} ( X ; {λ}_{i} , {μ}_{i} ) N ( X ) , \\ X ∈ {Γ}_{int} ( {θ}_{geo} ) . \qquad ( A20 )$$

Then, place residual points X_{Γ}⁽ⁱ⁾(θ_{geo}) (i∈{1,2, . . . , N_{Γ}}) on Γ(θ_{geo}), and construct the loss. component on the interface of two materials by taking a weighted sum as

$${int}_{} ( {λ}_{m} , {λ}_{i} , θ ) = {α}_{Dint} {Dint}_{} ( {λ}_{m} , {λ}_{i} , θ ) + {α}_{Nint} {Nint}_{} ( {λ}_{m} , {λ}_{i} , θ ) , \qquad ( A21 )$$

$$where$$

$${Dint}_{} ( {λ}_{m} , {λ}_{i} , θ ) = \frac{1}{{N}_{Γ}} {\sum }_{i = 1}^{{N}_{Γ}} {\vert {\tilde{r}}_{Dint} ( {X}_{Γ}^{( i )} ( {θ}_{geo} ) ; {λ}_{m} , {λ}_{i} ) \vert}^{2} \qquad ( A22 )$$

$${Nint}_{} ( {λ}_{m} , {λ}_{i} , θ ) = \frac{1}{{N}_{Γ}} {\sum }_{i = 1}^{{N}_{Γ}} {\vert {\tilde{r}}_{Nint} ( {X}_{Γ}^{( i )} ( {θ}_{geo} ) ; {λ}_{m} , {λ}_{i} , {μ}_{m} , {μ}_{i} ) \vert}^{2} . \qquad ( A23 )$$

Hence, the loss function in the case of multiple materials is the summation of the loss function for each single material and the additional loss [symbol] ᵢₙₜ for the material interface.

For forward problems with no unknown parameter (θᵤₙₖ=Ø, hence θ=Ø for convenience), measurement data may not be available. For incompressible materials, for example, the loss function can be written as a weighted sum of all the four loss terms that correspond to PDEs, the incompressibility condition, displacement boundary conditions, and traction boundary conditions, respectively:

$${for}_{} ( λ ) = {α}_{PDE} {PDE}_{} ( λ ) + {α}_{inc} {inc}_{} ( λ ) + {α}_{D} {D}_{} ( λ ) + {α}_{N} {N}_{} ( λ ) . \qquad ( A24 )$$

where α_{PDE}, α_{inc}, α_{D}, α_{N} are the weights of the loss components. Each loss term is defined by

$${PDE}_{} ( λ ) = \frac{1}{{N}_{Ω}} {\sum }_{i = 1}^{{N}_{Ω}} {\vert {\tilde{r}}_{PDE} ( {X}_{Ω}^{( i )} ; λ ) \vert}^{2} \qquad ( A 25 )$$

$${inc}_{} ( λ ) = \frac{1}{{N}_{Ω}} {\sum }_{i = 1}^{{N}_{Ω}} {\vert {\tilde{r}}_{inc} ( {X}_{Ω}^{( i )} ; λ ) \vert}^{2} \qquad ( A26 )$$

$${D}_{} ( λ ) = \frac{1}{{N}_{D}} {\sum }_{i = 1}^{{N}_{D}} {\vert {\tilde{r}}_{D} ( {X}_{D}^{( i )} ; λ ) \vert}^{2} \qquad ( A27 )$$

$${N}_{} ( λ ) = \frac{1}{{N}_{N}} {\sum }_{i = 1}^{{N}_{N}} {\vert {\tilde{r}}_{N} ( {X}_{N}^{( i )} ; λ ) \vert}^{2} . \qquad ( A28 )$$

The solution of forward problems using PINNs can be expressed as:

$$\hat{λ} = {arg min}_{λ} {for}_{} ( λ ) , \qquad ( A29 )$$

The displacement solved by the PINN is ũ(X; \hat{λ}) for X∈Ω.

According to the exemplary embodiments of the present disclosure, it is noted that for Case 0, the Young's modulus of the matrix is E=1.0, and the Poisson's ratio is v=0.3. For Cases 1, 2, 4 and 5, the shear modulus of the matrix (incompressible Neo-Hookean material) is μᵢ=0.333, and the external load is P₀=0.3. For Case 3, the parameters fed to the PINN include material parameters E=1.0, α=0.1, n=10, and σ_{Y}=0.005 and the external load P₀=0.002. For Cases 0, 1, 3, 4, 5, there are Nᵤ=1.0 measurement points of on each edge of the matrix. For Case 2, measurement points are inside the solid, with 10 of them uniformly placed on the line, X₁=−0.15, X₁=0.15, X₂=−0.45, X₂=0.45 respectively. For the modified Case 5 with additional measurements, the five additional measurement points are located at (−0.3,0.05), (−0.3,0.10), (−0.3,0.15) in the matrix and (−0.05,0.10), (0.15,0.10) in the inclusion. However, these are merely illustrative embodiments, and not meant to be comprehensive of all possibilities when considering identification problems utilizing the PINNs of the present disclosure.

The present disclosure can utilize TensorFlow 1.14 to build up our PINN. Each NN in the PINN has 4 hidden layers, each with 30 neurons. For Cases 0, 1, 2 and 4, there is one single NN in the PINN. There are two NNs in the PINN in Case 3 (one NN for (u₁, u₂), the other NN for (σ₁₁, σ₁₂, σ₂₂)) and Case 5 (one NN for each material). The present disclosure can adopt the layer-wise adaptive “tanh” function as the activation function for the NNs. Among the trainable parameters of the NN, weights are initialized with Xavier initialization, biases are initialized as zeros, and the variable of adaptive activation are initialized as ones. The illustrative example of the present disclosure utilizes the Adam optimizer to train the network. The learning rate is 0.001 for all cases except Cases 2 and 3.

For the illustrative embodiment in Case 2, the unknown geometric parameters directly defined in the code are the location of the center of the slit (X₁^{(c)}, X₂^{(c)}), half length of the slit L, and tilting angle of the slit Γ, which are post-processed to be the coordinates of the locations of the centers of the slit tips (X₁⁽¹⁾=X₁^{(c)}−L sin Γ, X₂⁽¹⁾=X₂^{(c)}+L cos Γ, X₁⁽²⁾=X₁^{(c)}+L sin Γ, X₂⁽²⁾=X₂^{(c)}−L cos Γ). The learning rate is 0.001 for Γ and 0.0002 for (X₁^{(c)}, X₂^{(c)}, L). For Case 3, the learning rate is 0.00005.

For the illustrative embodiments Cases 0, 1, 3 and 5, the assignments of residual points are similar. 3200 internal points for the PDE and incompressibility are assigned for the illustrative embodiment (800 points for each of the four sub-regions; 40 along the circumferential direction and 20 along the radial direction). For Case 5 only, 400 residual points are assigned in the inclusion material. The illustrative embodiment of the present disclosure places 160 residual points on the outer boundary for enforcing traction boundary conditions. Another 160 residual points are placed on the inner boundary of the matrix, which enforce the traction-free boundary conditions for Cases 1 and 3 and the interface conditions for Case 5. The points of displacement measurements are uniformly placed on the outer boundary of the matrix.

For the illustrative embodiment Case 2, the smallest length scale is determined by the W, which poses a limitation for the interval of residual points. To accurately resolve the displacement field, especially around the slit tip where the displacement changes drastically and the stress concentration exists, a large density of residual points may be needed in the computational domain. For this case, there are 49000 internal points, 700 points on the outer boundary, and 700 points on the inner boundary. For the illustrative embodiment Case 4, the square region can be divided into two parts, each with one void. For each part, residual points are assigned in a similar way to Case 1.

For the illustrative embodiments Cases 0-5, the weights of the loss terms in Eq. A13 to be α_{PDE}=α_{inc}=α_{D}=αᵤ=1 are designated when applicable. The weight for traction boundary conditions α_{N} is also set to be 1, but the present disclosure evaluates this part separately for each of the five boundaries (left, right, top, bottom, inner), leading to five loss terms related to traction boundaries, each with weight 1, in the total loss. In Case 2, the calculation of the mean squared error of the PDE loss term [symbol] _{PDE}(λ, θ) is further weighted inversely by the local density of residual points, so that different spatial regions contribute equally to the loss term of PDEs.

For illustrative embodiments Cases 0 and 3, the small values of loading (roughly O(10⁻³)) cause difficulties in training the PINN. To mitigate this issue, the external loading (and σ_{Y}=0.005 in Case 3) can be scaled up 100 times and feed the scaled loading into the PINN. According to Eqs. A15 and A16, such scaling only results in 100 times the original displacement (and hence strain) and does not essentially changes the characteristics of the solution. After obtaining the solution from the PINN, the displacement/strain solution can be scaled back (0.01 times) to its original value.

Illustrative embodiments of the present disclosure may use Abaqus as the FEM solver to generate the displacement data {u*⁽ⁱ⁾}ᵢ₌₁^{N}^{ᵤ} and the entire displacement field, given the reference values of unknown parameters θ*ᵤₙₖ. The linear density of meshes is 120 per unit length, which can be sufficiently dense so that the FEM/Abaqus solution is accurate enough to serve as the reference solution. Plane-strain quadratic elements with hybrid formation (CPE8H elements) can be applied for hyperelasticity.

According to exemplary embodiments of the present disclosure, a simple comparison on the model performance in terms of accuracy and efficiency with different strategies on optimizers based on Case 4. In the main text, the Adam optimizer can be adopted as the only optimizer throughout the entire training process with 1M iterations (called strategy 1A in this section), after which both the parameter estimations and loss function reach a relative plateau. Such a strategy gives high accuracy and can help facilitate study the convergence history as a fundamental characteristic of our method. To achieve a reasonable accuracy practically, one may not need as many as 1M iterations. Here, the present disclosure considers training the PINN over 200K iterations only (called strategy 1B). To further improve the computational efficiency, it is also contemplated that one may consider using Adam for the first few iterations (40K iterations in our case), and then switching to L-BFGS (48) (called strategy 2), which is common for training PINNs practically. Then, one can compare the results of the three strategies (1A, 1B and 2) in view 220 of FIG. 22 in terms of accuracy of parameter estimation and computational efficiency. The computational time can be measured by running the code on typical machines using CPU only. The results of strategy 1B indicates that training the PINN with 200K iterations provides reasonably high accuracy. Strategy 2 using L-BFGS performs even better. The accuracy is similar to strategy 1A is achieved, while the computational cost may be significantly reduced.

The present disclosure demonstrates the capability of the new method based on PINNs in effectively solving inverse problems in solid mechanics involving parameterized geometry. Specifically, the present disclosure focuses on the analyses of internal structures and defects in engineering solids for two types of problems: (1) geometry (and material) identification; (2) design of optimized and/or improved geometry. Using illustrative examples, this disclosure demonstrates that the presented method works for different behavior of the solid material, including (1) linear elasticity, (2) hyperelasticity, and (3) deformation plasticity. The present disclosure demonstrates that, for identification problems, the present framework is able to accurately estimate the unknown geometric and material parameters with a relative error O(10⁻²) when proper displacement data are supplied to ensure identifiability. For design problems, the present disclosure demonstrates that the presented method is able to design optimized and/or improved geometry with a relative error of O(10⁻³) compared with reference optimal design. In addition to identifying the unknown geometry or optimal geometry, the present disclosure demonstrates the capability of the method in reconstructing the full-field mechanical quantities corresponding to the identified/designed geometry, such as displacement, strain, and stress.

According to the exemplary embodiments described herein in detail, the approach presented in the present disclosure possesses some unique characteristics, endowing this method with some distinct advantages. It is contemplated that the method inherits and extends the applicability and advantages of original PINNs. It is contemplated that the present disclosure provides a unified framework for solving forward problems and inverse problems with unknown parameters in PDEs (material identification) and/or domains (geometry identification; geometry design. Unlike traditional methods based on FEM, there is neither the need to design problem-specific algorithms to update estimated unknown parameters beyond the forward solver, nor the need to repeatedly re-mesh the computational domain throughout the iterations in the present disclosure. In the present disclosure, the update of geometry is realized by the automated process built in the deep learning algorithms. In particular, the estimation of geometric parameters is automatically updated as the PINN seeks to minimize the loss function through the iterative training process. With open-source deep learning libraries (e.g., TensorFlow, Pytorch), the entire length of the PINN code of the present disclosure for the current work is merely a few hundred lines. From the perspective of both design and implementation of the algorithm, PINNs significantly reduce the human effort and related costs in setting up algorithms for inverse problems. Diverse types of conditions in the problem definition can be enforced and integrated into the training guideline of the PINN in the form of loss function. For example, displacement and traction boundary conditions or data (for identification problems) can be accurately enforced in the training process; maximum stiffness or minimum principal stress as the objective function (for design problems) can be integrated into the training guideline within the PINN framework. On the other hand, compared to typical data-driven deep learning approaches, PINNs have the advantage of utilizing well-established mechanics formulations as training guidelines, thereby requiring data only for the current instance of the problem setup and ensuring data-efficiency. It is noted that data-driven deep learning methods, on the other hand, require a large dataset containing numerous problem setups.

It is noted that, in accordance with exemplary embodiments, the present disclosure adopts the Adam optimizer as the optimization algorithm to achieve best accuracy and to study the convergence history as a fundamental characteristic of our method. The PINN is trained until both loss function and the estimated parameters reach a relative plateau. With such a setup, the computational time for Case 4 of identification, for example, is around 11 hours on a typical machine (with CPU only) to complete the entire 1M iterations and achieve high accuracy. It is contemplated by the present disclosure that reasonable accuracy has been achieved within the first 200K iterations. One may further combine Adam and the L-BFGS optimizer to achieve similar accuracy within much less computational time (around 30 minutes; see Paragraph 000105 and FIG. 22 for detailed results). Recently, parallel PINNs have been proposed to accelerate the learning process of PINNs by utilizing multiple CPUs and GPUs and introducing parallel algorithms. In addition, other studies have focused on analyzing convergence rate of PINNs and proposing practical techniques for accelerating convergence. With the ongoing efforts to improve the original formulation of PINNs, the computational efficiency is expected to be significantly enhanced over time.

The present disclosure focuses on the matrix-void/inclusion system in Cases 0-5 as a simple proof of concept for identification problems, seeking to characterize the internal structures with static loading on outer boundaries. According to Saint-Venant's principle, under static loading, the inhomogeneous stress and deformation states caused by the internal void/inclusion decays as the distance from the void/inclusion increases. Subsequently, the measurements on outer boundaries essentially provide the PINN with limited amount of information regarding the internal void/inclusion. Modern experimental techniques have adopted dynamic external loading such as ultrasound to acquire time-dependent measurements, through which it is anticipated that the performance of the method of the present disclosure will benefit from more information provided by measurements.

The applications presented in this disclosure can involve a wide range of engineering problems. Geometry design problems are prevalent in mechanical, civil and material engineering, where one needs to design the geometry, topology, and/or internal structures of materials/structures for achieving optimized and/or improved performance based on certain objectives as guidelines. Geometry identification problems are closed related to defect detection, which represents a broad class of practical engineering needs in various fields, where identification and characterization of internal structures and defects in materials are essential. Illustrative embodiments of the present disclosure include experimental techniques, which have so far been developed for different materials based on ultrasound, active thermography, eddy current, optical coherent tomography, and microwave. By integrating the respective physical principles in these problems, it is contemplated that the approach of the present disclosure can be combined with these techniques for dealing with unknown/moving geometries, which extends our method beyond continuum solid mechanics.

Embodiments of the above-described systems and methods can be implemented in digital electronic circuitry, in computer hardware, firmware, software and combinations thereof. The implementation can be as a computer program product. The implementation can, for example, be in a machine-readable storage device, for execution by, or to control the operation of, data processing apparatus. The implementation can, for example, be a programmable processor, a computer, and/or multiple computers.

A computer program is provided in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose logic circuitry. The circuitry can, for example, be a FPGA (field programmable gate array) and/or an ASIC (application-specific integrated circuit). Subroutines and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). The mobile computing device includes, for example, a smartphone or tablet (e.g., iPhone®, iPad®, Android® device, Windows Phone®, etc.).

The terms comprise, include, and/or plural forms of each are open ended and include the listed parts and can include additional parts that are not listed. The term and/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

Although various aspects of the present disclosure are described herein in terms of various exemplary embodiments, it should be understood that variations and modifications may be made to the disclosure described herein to adopt it to various usages and conditions within the scope of applicant's invention as claimed herein.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for analyzing an aspect of a solid material/structure, comprising:
receiving one or more geometric variables as one or more inputs to physics-informed neural networks (PINNs);
characterizing/parametrizing a first geometry according to the one or more geometric variables; and
identifying one or more aspects of the solid material/structure based on the first geometry,
wherein characterizing/parametrizing a first geometry is performed in a trainable manner.

2. A method for analyzing an aspect of a solid material/solid structure, comprising:
receiving one or more geometric variables as one or more inputs to physics-informed neural networks (PINNs); and
generating features and parameters of a second geometry of one or more aspects of a solid material,
wherein generating features and parameters of a second geometry achieves an improved performance based on one or more objectives,
wherein an improved performance based on one or more objectives is performed in a trainable manner.

3. The method of claim 1, wherein the one or more aspects of the solid material/structure includes at least one of one or more internal structures, internal surfaces/boundaries, external structures, or external surfaces/boundaries.

4. The method of claim 1, wherein the one or more aspects of the solid/structure includes one or more defects in the solid material/structure.

5. The method of claim 1, wherein the trainable manner includes substituting one or more geometric variables such as geometry trainable variables, geometry-dependent training points, and/or making the gradient with respect to geometry tractable.

6. The method of claim 1, wherein characterizing/parametrizing a first geometry includes inversely characterizing a first geometry of at least one of one or more internal structures, internal surfaces/boundaries, external structures, or external surfaces/boundaries according to displacement data.

7. The method of claim 2, wherein generating features and parameters of a second geometry includes inversely designing the second geometry of at least one of internal structures, internal surfaces/boundaries, external structures, or external surfaces/boundaries according to a predefined objective function.

8. The method of claim 1, wherein identifying one or more aspects of the solid material/structure based on the first geometry includes concurrently identifying one or more full field stresses, strains, and/or displacements in the one or more aspects of the solid material/structure.

9. The method of claim 1, wherein a framework for inversely characterizing and/or designing involves unknown/moving domains directly parameterizing a computational domain/geometry with material and geometry parameterization.

10. The method of claim 6, wherein inversely characterizing a first geometry includes minimizing a discrepancy/loss between the displacement data and one or more results of a forward solver.

11. The method of claim 7, wherein inversely designing a second geometry includes minimizing the objective function.

12. The method of claim 1, wherein characterizing/parametrizing a first geometry or generating features and parameters of a second geometry includes representing at least one of one or more internal structures, internal surfaces/boundaries, external structures, or external surfaces/boundaries by analytical function(s), parameterized function(s), a non-uniform rational basis spline (NURBS) or other neural network(s),
wherein a shape of the at least one of one or more internal structures, internal surfaces/boundaries, external structures, or external surfaces/boundaries are simple or arbitrarily complicated.

13. The method of claim 5, wherein concurrently identifying the full field stresses, strains, displacements in the one or more solid materials/structures includes one or more different shapes and/or topologies of the one or more solid materials/structures and different constitutive models for describing the mechanical properties of the one or more solid materials/structures,
wherein the different constitutive models for describing the mechanical properties of the one or more solid materials/structures include measuring linear elasticity, nonlinear elasticity or hyperelasticity, and plasticity.

14. The method of claim 1, wherein the trainable manner includes a pretraining process for the PINNs, comprising:
maintaining one or more estimated unknown parameters θ defined as fixed/not trainable and updating one or more trainable parameters of a neural network (NN) λ for one or more iterations;
solving one or more forward problems to capture a qualitative pattern of a displacement field and a stress field; and
solving one or more forward problems until both a loss function and one or more estimated geometric parameters reach a relative plateau following pretraining of the PINNs,
wherein both λ and θ are then trainable,
wherein λ converged towards to a desired local minimum,
wherein the pretraining process stabilizes the trainable manner of the PINNs.

15. The method of claim 14, wherein an estimation of geometric parameters is automatically updated as the PINNs minimize the loss function during the pretraining and/or training process for the PINNs,
wherein automatically updating includes enforcing one or more diverse types of conditions in problem definition for integration into the PINNs in the form of the loss function during the pretraining and/or training process for the PINNs.

16. The method of claim 3, wherein the one or more geometric variables parameterize the computational domains of partial differential equations (PDEs) and boundary conditions,
wherein the one or more geometric variables are first defined as trainable before expressing one or more locations of residual points as functions of the one or more geometric variables.

17. The method of claim 16, wherein the one or more locations of the residual points are automatically updated as an estimation of the one or more geometric variables are updated throughout the training process,
wherein the one or more locations of the residual points for one or more different conditions are in their correct domains, allowing the capturing of a gradient of a loss function [symbol] with respect to the one or more geometric variables,
wherein the residual points for the one or more different conditions allows for the PINNs to correctly update estimation and/or design of the one or more geometric variables throughout the training process and characterize/design the at least one of one or more internal structures, internal surfaces/boundaries, external structures, external surfaces/boundaries and/or defects.

18. The method of claim 1, wherein characterizing/parametrizing a first geometry includes accurately estimating the one or more geometric variables and one or more material parameters with limited non-destructive measurements,
wherein accurately estimating the unknown geometric and one or more material parameters includes a relative error O(10⁻²) when proper displacement data are supplied to ensure identifiability,
wherein characterizing/parametrizing a first geometry further includes placing one or more displacement measurement points only on a boundary of a solid material/structure.

19. A method for utilizing physics-informed neural networks (PINNs) to examine internal structures and defects of solid materials/structures according to claim 1, comprising:
applying a neural network to approximate the primary solution fields;
integrating one or more mechanical laws into the PINN by deriving relevant mechanical quantities of interest from one or more neural network (NN) outputs, such as strain, stress, and/or residual of equilibrium partial differential equations (PDEs);
formulating a loss function [symbol] (λ, θ), wherein the loss function [symbol] (λ, θ) measures a discrepancy between predicted mechanical quantities of interest and their respective true values provided by the one or more mechanical laws and measured data; and
conducting parameter estimation through a training of the PINN, wherein the training of the PINN includes updating/training unknown parameters θ=(θₘₐₜ, θ_{geo}) and neural networks parameters λ to minimize the loss function.

20. A method for utilizing physics-informed neural networks (PINNs) to examine internal or external structures and defects of solid materials/structures according to claim 2, comprising:
minimizing [symbol] _{PDE}(λ, θ), [symbol] _{BC}(λ, θ), wherein minimizing [symbol] _{PDE}(λ, θ), [symbol] _{BC}(λ, θ) includes satisfying a governing partial differential equation (PDE) and one or more boundary conditions as the PINN seeks to minimize a loss function,
wherein one or more constraints are satisfied through minimizing [symbol] _{cnstr}(λ, θ), wherein the one or more constraints are directly incorporated through designing an architecture of PINNs,
wherein a design target is achieved through minimizing [symbol] _{target}(λ, θ), and
wherein one or more relevant geometric parameters in θ are adjusted to minimize the loss function and realize a design of optimal geometry.
